(19) United States
(12) Patent Application Publication
(10) Pub. No.: US 2005/0214333 A1
(43) Pub. Date: Sep. 29, 2005

(54) Cosmetic with sensitive ingredients

(75) Inventors: Ghita Lanzendoerfer, Hamburg (DE); Hiedi Riedel, Hamburg (DE); Stephan Ruppert, Hamburg (DE); Lorenz Eckers, Tostedt (DE); Volker Kallmayer, Hamburg (DE)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 10/991,329

(22) Filed: Nov. 17, 2004

(30) Foreign Application Priority Data
Nov. 17, 2003 (DE) 103 54 052.0

(51) Int. Cl. A61K 7/00; G01F 11/00

(52) U.S. Cl. 424/401; 222/216

(57) ABSTRACT

The present invention relates to a cosmetic preparation, a dispenser for cosmetic preparations with a substantially cylindrical container accommodating the cosmetic preparation, which includes on the side toward its bottom a follow-up plunger that is adapted for sliding displacement along an inner wall of the container under the pressure of the ambient atmosphere, and which mounts at its upper end a head section that is adapted for sliding displacement relative to the container, with the head section including a dispensing channel for the product that can be connected to the container in a communicating manner, and acting upon a manually actuatable delivery device with a volume variable delivery chamber for the product.

The present invention relates to a cosmetic which is composed of a dispenser and a preparation with sensitive ingredients.

The desire to look beautiful and attractive is rooted in humans by nature. Even when the ideal of beauty has undergone changes in the course of time, the endeavor for a flawless exterior has always been a goal of humans. A substantial part of a beautiful and attractive exterior is formed in this connection by the condition and the appearance of the skin and the skin appendages.

In order that the skin and the skin appendages, these include primarily hair and nails, are able to fulfill their biological functions to the full extent, they need regular cleansing and care as well as protection against UV radiation. In this connection, cleansing serves the purposes of removing dirt, sweat, and remnants of deceased body cells, which form an ideal culture medium for all kinds of infectious germs and parasites. Cosmetic cleansing products are normally offered in the form of gels, lotions, and solids (soap bars, syndet soaps). Skin care products, normally creams, ointments, or lotions are used in most cases to moisturize and refat the skin as well as to supply the skin with cosmetic or dermatological agents (for example, vitamins, antioxidants, UV light protection filters). This will be important just when the natural regeneration capacity of the skin does not suffice to make up for stresses and damage to the skin which result from environmental influences, mechanical or chemical irritations, sun light and wind (and which accelerate, among other things, aging of the skin).

Depending on viscosity and purpose of application, conventional cosmetic preparations are stored and presented in different packaging means. In addition to the long-time known tubes, bottles, aerosol sprays, etc., mechanical pump dispensers have also been used to an increasing extent in more recent times. They permit applying not only low-viscosity, aqueous, or aqueous alcohol-containing preparations, but are also used more frequently for applying/dosing milks and lotions on the basis of emulsions.

For example, in comparison with bottles and tubes, pump dispensers as means for storing and applying cosmetic and/or dermatological preparations have the advantage that depending on the delivery volume of the pump of the dispenser, they allow to dose the preparations in a very simple manner, so that the quantity of the preparation to be removed can already be adjusted by the manufacturer to the quantity required for the application. With that, it is possible to avoid overdosages and underdosages by the user.

A further advantage of pump dispensers over conventional packaging means lies in the fact that they permit removing the preparations from the storage container in a relatively clean and hygienic way. A direct contact of the preparation remaining in the storage container with body surfaces (for example, hands) and a thereby produced contamination with germs (for example, bacteria, fungi, etc.) is effectively avoided, since the preparation is forced out of the storage container under pressure.

In comparison with aerosol cans that have been known for years, pump dispensers have the great advantage that they totally avoid the hazard potential that results from possible damage to a pressurized gas container (aerosol can). Since pump dispensers do without the use of combustible propellants as the delivery medium, which are harmful to the ozone layer, their use is especially safe and environment-friendly. Last but not least, pump dispensers can be produced and discarded in a more cost-favorable and environmentally protective manner.

For this reason, a plurality of mechanical pump dispensers (also named dispensers within the scope of the present specification) have been developed for cosmetic preparations in the past. These dispensers also permit applying formulations of a higher viscosity.

Thus, dispensers with a slidable follow-up plunger and a manually actuatable delivery device with a volume-variable delivery chamber are known as movable storage containers in a plurality of examples of application, for example, for the care of the body, in the medicine for the application of medications, or also in commerce for the preparation of pasty foods. Accordingly diverse is also the configuration of the dispensers in use for making available the great variety of pasty substances, primarily with respect to their direct delivery and operating mechanism.

A dispenser of this type is disclosed, for example, in EP-A-0 230 252. In this known dispenser, the manually actuatable delivery device comprises a delivery plunger which permits varying the volume of the delivery chamber. The delivery plunger engages a tubular section that is made integral with the head section. When using the dispenser, the head section is displaced by manual actuation from a starting position in the axial direction toward the container.

This displacement leads directly to a sliding movement of the delivery plunger along the inner wall of the delivery chamber while reducing the volume thereof. The internal pressure that builds up in the delivery chamber causes at first a nonreturn valve to open which is formed in the delivery plunger and covers a passageway of the delivery plunger in the shape of an ellipse. This nonreturn valve then delivers the pasty product during a further reduction of the volume of the delivery chamber in the direction toward a dispensing channel for removal from a product discharge opening that is formed on the head section.

In the case of the known dispenser, it is necessary in a first step to build in the delivery chamber an internal pressure that overcomes the closing forces of the nonreturn valve.

Likewise, the delivery of the pasty substance by the nonreturn valve leads to a pressure loss, which is disadvantageous, inasmuch as for compensating this pressure loss, it is necessary to apply increased forces of pressure for an axial displacement of the head section in the direction toward the container.

The publication WO 03/004374 discloses a dispenser that is improved to the extent that it permits an actuation with lesser forces. In this dispenser, the delivery device comprises a delivery element that is adapted for axial displacement relative the container and the head section. The delivery element comprises a plunger that is adapted for sliding displacement in the delivery chamber. The delivery plunger connects to a delivery shaft that circumferentially surrounds a delivery channel which comprises a delivery channel inlet opening communicating with the delivery chamber and a delivery channel outlet opening. By displacing the delivery element relative to the head section, the channel outlet opening can be moved to a position, in which it opens toward the dispensing channel.

Mechanical pump dispensers of the art further have the following disadvantages:

The cosmetic preparation in the storage container is not adequately shielded against the environment. The volume of the preparation that is removed from the storage container during the application of the cosmetic must be replaced with ambient air. This causes humidity (water) and oxygen to enter the storage container and to come into contact with the preparation. This entry of air into the storage container cannot be avoided in any pump dispenser. For the preparation stored in the container, the contact with air will be problematic especially when the formulation contains one of more ingredients (in particular active agents), which
are sensitive to oxidation with oxygen or air, and/or
sensitive to hydrolysis with water or moisture of the air;
cause a temperature change of the preparation by the action of water, air moisture and/or oxygen;
lose their physiological effectiveness by the action of water, air moisture, and/or oxygen; and/or
irritate the olfactory receptors of humans and/or animals, for example, which contain fragrances and/or perfumes, and/or repellents.

If a cosmetic or dermatological preparation contains at least one ingredient of this kind, it will lose and/or increasingly change over a longer period of storage in the pump dispenser its effectiveness or its composition that has been predetermined by the manufacturer.

By experience, the quantities of air, i.e., in particular oxygen and water that enter the storage container as a result of a volume exchange, play a subordinate role in comparison with a contamination by air or oxygen and water, which results from the fact that the storage container is not adequately protected against the environment. Especially critical is the outlet opening, since at this point the product is exposed to the surroundings without protection. In the extreme case, the user will be unable to receive a product that has not been decomposed, since the accumulating product volume that is to be removed next is always subjected to the greatest exposure to ambient oxygen or moisture of the air. Likewise plugs that are to close the outlet opening after use, do not adequately solve the problem, because even when used, the product will always be squeezed out to the sides. A lasting (continuous) contact with “fresh” oxygen or water from the ambient air results also in the storage container over a longer period in a loss of effectiveness or in a changed composition of the preparation. For this reason, the pump dispensers of the art cannot be used for preparations with the aforesaid ingredients.

It is therefore an object of the present invention to eliminate the deficiencies of the prior art and to develop cosmetics which do not have the described deficiencies or exhibit them only to a small extent. In particular, it is intended to develop a cosmetic that permits storing and dispensing in a constant quality with the aid of a mechanical pump dispenser also preparations containing ingredients that are sensitive to oxidation and/or humidity.

It is a further object of the present invention to improve a dispenser of the initially described type to the extent that it permits actuation with lesser forces, and moreover that it prevents a pasty product to be dispensed from being adversely affected by oxidation.

Surprisingly, the objects are accomplished by a cosmetic from a) a dispenser for pasty products with a substantially cylindrical container (1) accommodating a cosmetic and/or dermatological preparation, which comprises on the side of its bottom a follow-up plunger (22) that is adapted for sliding displacement along the inner wall of the container under the pressure of the ambient atmosphere, and which mounts at its upper end a head section (3) adapted for sliding displacement relative to the container (1), with the head section (3) comprising a dispensing channel (32) for the product, that can be connected in a communicating manner to the container (1), and acting upon a manually actuatable delivery device with a volume-variable delivery chamber (100) for the product, characterized in that the delivery device comprises a delivery element (5) adapted for axial displacement relative to the container (1) and the head section (3), the delivery element (5) including a delivery plunger (51) that is adapted for sliding displacement in the delivery chamber (100) and connects to a delivery shaft (50), with the delivery shaft circumferentially surrounding a delivery channel (50a), which comprises a delivery channel inlet opening (53) communicating with the delivery chamber (100) and a delivery channel outlet opening (58), the delivery channel outlet opening (58) being movable by displacing the delivery element (5) relative to the head section (3) to a position, in which the delivery channel outlet opening (58) opens toward the dispensing channel (32); and
b) a cosmetic preparation containing one or more ingredients, which have one or more of the following properties, namely: which I) are sensitive to oxidation with oxygen or air; II) sensitive to hydrolysis with water or air moisture; III) irritate the olfactory receptors of humans and/or animals; IV cause a temperature change of the preparation under the influence of water, air moisture and/or oxygen; and V) lose their physiological effectiveness under the influence of water, air moisture, and/or oxygen.

Furthermore, the objects are surprisingly accomplished by the use of a dispenser for pasty products with a substantially cylindrical container (1) accommodating a cosmetic and/or dermatological preparation, which comprises on the side of its bottom a follow-up plunger (22) that is adapted for sliding displacement along the inner wall of the container under the pressure of the ambient atmosphere, and which mounts at its upper end a head section (3) adapted for sliding displacement relative to the container (1), with the head section (3) comprising a dispensing channel (32) for the product, that can be connected in a communicating manner to the container (1), and acting upon a manually actuatable delivery device with a volume-variable delivery chamber (100) for the product, characterized in that the delivery device comprises a delivery element (5) adapted for axial displacement relative to the container (1) and the head section (3), the delivery element (5) including a delivery plunger (51) that is adapted for sliding displacement in the delivery chamber (100) and connects to a delivery shaft (50), with the delivery shaft circumferentially surrounding a delivery channel (50a), which comprises a delivery channel inlet opening (53) communicating with the delivery chamber (100) and a delivery channel outlet opening (58), the delivery channel outlet opening (58) being movable by displacing the delivery element (5) relative to the head section (3) to a position, in which the delivery channel outlet opening (58) opens toward the dispensing channel (32)
for storing and dispensing cosmetic and/or dermatological preparations containing one or more ingredients, which have one or more of the following properties, namely: which I) are sensitive to oxidation with oxygen or air; II) sensitive to hydrolysis with water or air moisture; III) irritate the olfactory receptors of humans and/or animals; IV cause a temperature change of the preparation under the influence of water, air moisture and/or oxygen; and V) lose their physiological effectiveness under the influence of water, air moisture, and/or oxygen.

After removing the product from the described dispensers, the delivery channel outlet opening is advantageously closed in accordance with the invention, with the filled product being not forced outwardly but inwardly and thus being protected against the environment.

Within the present disclosure, the term “according to the invention” relates both to the cosmetic, the dispenser, and the preparation per se, and to the uses thereof.

Within the scope of the present disclosure, the pasty preparation represents according to the invention a preparation of a viscosity from 500 to 20,000 mPas (measured with the aid of a viscometer of the type VT O₂ Viscotester by Haake with the following measuring parameters: temperature 25° C., rotational body diameter 24 mm, rotor speed 62.5 rpm).

It also came as a surprise for the person of skill in the art, that it was possible to protect the preparations of the invention with a reduced quantity of preservatives in an effective manner against infestation by microorganisms.

According to the invention, the cosmetic of the invention or the dispenser used in accordance with the invention contains in the preparation one or more ingredients, which have one or more of the following properties, namely: which I) are sensitive to oxidation with oxygen or air; II) sensitive to hydrolysis with water or air moisture; III) irritate the olfactory receptors of humans and/or animals; IV cause a temperature change of the preparation under the influence of water, air moisture and/or oxygen; and V) lose their physiological effectiveness under the influence of water, air moisture, and/or oxygen, each in a total concentration from 0.01 to 5 wt. %, preferably in a concentration from 0.05 to 3 wt. %, and more preferably in a concentration from 0.1 to 1 wt. %, each based on the total weight of the preparation.

In accordance with the invention the cosmetic of the invention or the dispenser used in accordance with the invention is advantageously characterized in that one or more ingredients which have one or more of the following properties, namely: which I) are sensitive to oxidation with oxygen or air; II) sensitive to hydrolysis with water or air moisture; III) irritate the olfactory receptors of humans and/or animals; IV cause a temperature change of the preparation under the influence of water, air moisture and/or oxygen; and V) lose their physiological effectiveness under the influence of water, air moisture, and/or oxygen, are selected from the group of antioxidants, vitamins, enzymes, coenzymes, perfumes, fragrances, repellents, selftanners, unsaturated lipids, oils, fats, waxes, polyphenols, enzyme, flavonoids and isoflavonoids, lignans, polyols, polyethylene glycols, as well as plant extracts, which contain one or more of the foregoing active ingredients.

In this connection, it is preferred in accordance with the invention that the cosmetic or the dispenser of the invention contains in the preparation one or more ingredients selected from the groups consisting of vitamin A and its derivatives, vitamin B and its derivatives, vitamin C and its derivatives, vitamin E and its derivatives; vitamin F; polyphenols, ubiquinone Q10 (also in its reduced form); 2,6-ditert.-butyl-4-methylphenol; dihydroxy acetone; niacinamide; pantothenic acid and its salts; panthenol; gamma-oryzanol; biotin; creatine, creatinine; subtilisin; alpha-glycosylrutin; allantoin; tannin; azulen; bisabolol; glycyrrhizin; hamamelin; urea; genistin; genistein; daidzin; daidzein; carnitine and its derivatives, and octadecene dicarboxylic acid as active ingredients, which have one of more of the following properties, namely: which I) are sensitive to oxidation with oxygen or air; II) sensitive to hydrolysis with water or air moisture; III) irritate the olfactory receptors of humans and/or animals; IV cause a temperature change of the preparation under the influence of water, air moisture and/or oxygen; and V) lose their physiological effectiveness under the influence of water, air moisture, and/or oxygen.

I. The Preparation of the Invention

According to the invention, it is advantageous that the cosmetic of the invention or the dispenser used according to the invention also contains in the preparation one or more of the following ingredients, which depending on their chemical nature may likewise fall under the active ingredients of the invention:

As advantageous UV light protection filters one may use according to the invention, for example, the following compounds:

Inorganic Pigments

Preferred inorganic pigments are metal oxides and/or other metal compounds that are sparingly soluble or insoluble in water, in particular the oxides of titanium (TiO₂), zinc (ZnO), iron (for example, Fe₂O₃), zirconium (ZrO₂), silicon (SiO₂), manganese (for example, MnO), aluminum (Al₂O₃), cerium (for example (Ce₂O₃), mixed oxides of the corresponding metals, as well as mixtures of such oxides, as well as the sulfate of barium (BaSO₄).

The titanium dioxide pigments may be present in the crystalline modification rutile as well as anatase. For the purposes of the present invention, they may advantageously be surface treated (“coated”). In this process, it is intended to form or maintain, for example, a hydrophilic, amphiphilic, or hydrophobic character. This surface treatment may consist in that the pigments are provided by methods known per se with a thin hydrophilic and/or hydrophobic, inorganic and/or organic coating. For the purposes of the present invention, the different surface coatings may also contain water.

The foregoing coated or uncoated titanium dioxides may also be used for the purposes of the present invention in the form of commercially available, oily or aqueous predispersions. It is possible and advantageous to add to these predispersions dispersants and/or solubilization agents.

The titanium dioxides of the invention are characterized by a particle size from 10 nm to 150 nm.

| | | Additional | |
|---|---|---|---|
| | | Constituents | |
| | | of the Pre- | |
| Trade name | Coating | dispersion | Manufacturer |
| MT-100TV | Aluminum hydroxide | — | Tayca |
| | stearic acid | | Corporation |
| MT-100Z | Aluminum hydroxide | — | Tayca |
| | stearic acid | | Corporation |
| MT-100F | Stearic acid | — | Tayca |
| | iron oxide | | Corporation |
| MT-500SAS | Alumina, silica | — | Tayca |
| | silicone | | Corporation |
| MT-100AQ | Silica | — | Tayca |
| | aluminum hydroxide | | Corporation |
| | alginic acid | | |
| Eusolex T-2000 | Alumina | — | Merck KgaA |
| | simethicone | | |
| Eosolex TS | Alumina, stearic acid | — | Merck KgaA |
| Titanium dioxide | None | — | Degussa |
| P25 | | | |
| Titanium dioxide | Octyltrimethylsilane | — | Degussa |
| T805 | | | |
| (Uvinul TiO₂) | | | |
| UV-Titanium | Alumina | — | Kemira |
| X170 | dimethicone | | |
| UV-Titan X161 | Alumina, silica | — | Kemira |
| | stearic acid | | |
| Tioveil AQ | Alumina | Water | Solaveil |
| 10PG | silica | propylene | Uniquema |
| | | glycol | |
| Mirasun TiW 60 | Alumina | Water | Rhone-Poulenc |
| | silica | | |

For the purposes of the present invention, specially preferred titanium dioxides are MT-100Z and MT-100 TV from Tayca Corporation, Eusolex T-2000 and Eusolex TS from Merck, and titanium dioxide T 805 from Degussa.

Zinc oxides for the purposes of the present invention may also be used in the form of commercially available, oily or aqueous predispersions. In accordance with the invention, suitable zinc oxide particles and predispersions of zinc oxide particles are characterized by a primary particle size of <300 nm, and are available from the listed manufacturers under the following trade names:

| Trade name | Coating | Manufacturer |
|---|---|---|
| Z- Cote HP1 | 2 wt.-% Dimethicone | BASF |
| Z- Cote | / | BASF |
| ZnO NDM | 5 wt.-% Dimethicone | H&R |
| MZ 707M | 7 wt.-% Dimethicone | M. Tayca Corp. |
| Nanox 500 | / | Elementis |
| ZnO Neutral | / | H&R |

Specially preferred zinc oxides for the purposes of the present invention are Z-Cote HP1 from BASF and the zinc oxide NDM from Haarmann & Reimer.

The total quantity of one or more inorganic pigments in the finished cosmetic preparation is advantageously selected from a range of 0.1 wt. % to 25 wt. %, preferably 0.5 wt. % to 18 wt. %.

Organic Pigments

An advantageous organic pigment for the purposes of the present invention is 2,2′-methylene-bis-(6-(2-benzotriazole-2-yl-4-(1,1,3,3-tetramethylbutyl)-phenol) [INCl: bisoctyltriazole], which is available from CIBA-Chemikalien GmbH under the trade name Tinosorb® M.

Further UV Light Protection Filters

Advantageous UV-A filter substances for the purposes of the present invention are dibenzoylmethane derivatives, in particular 4-(ter.-butyl)-4′-methoxydibenzoylmethane (CAS No. 70356-09-1), which is sold by Givaudan under the trademark Parsol® 1789 and by Merck under the trade name Eusolex® 9020.

Further advantageous UV-A filter substances are phenylene-1,4-bis-(2-benzimidazyl)-3,3′-5,5′-tetrasulfonic acid and its salts, in particular the corresponding sodium, potassium, or triethanolammonium salts, in particular phenylene-1,4-bis(2-benzimidazyl)-3,3′-5,5′-tetrasulfonic acid-bis-sodium salt with the INCl name Bismidazylate, which is available from Haarmann & Reimer under the trade name Neo Heliopan AP.

Also advantageous are 1,4-di(2-oxo-10-sulfo-3-bornylidene methyl)benzene and its salts (in particular the corresponding 10-sulfato compounds, in particular the corresponding sodium, potassium, or triethanolammonium salt), which is also referred to as benzene-1,4-di(oxo-3-bornylidenemethyl-10-sulfonic acid).

Further advantageous UV-A filter substances are hydroxygenzophenones, which are characterized by the following structural formula: 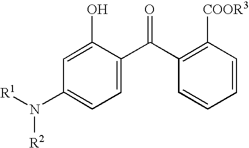
where
R¹ and R² are independently of each other hydrogen, C₁-C₂₀-alkyl, C₃-C₁₀-cycloalkyl or C₃-C₁₀-cycloalkenyl, with the substituents R¹ and R² being able to form a 5- or 6-member ring together with the nitrogen atom to which they are linked, and
R³ is a C₁-C₂₀ alkyl residue.

A particularly advantageous hydroxybenzophenone for the purposes of the present invention is 2-(4′-diethylamino-2′-hydroxybenzoyl)benzoic acid hexyl ester (also known as: aminobenzophenone), which is available from BASF under the trade name Uvinul A Plus.

Advantageous UV filter substances for the purposes of the present invention further include so-called broadband filters, i.e. filter substances which absorb both UV-A and UV-B radiation.

Advantageous broadband filters or UV-B filter substances are, for example, bis-resorcinyltriazine derivatives with the following structure: 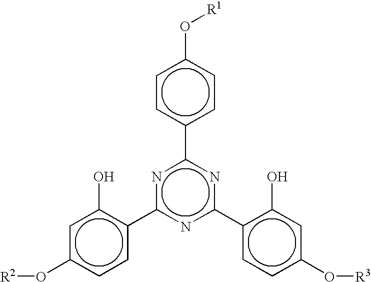
where R¹, R², and R³ are selected independently of one another from the group of branched and unbranched alkyl groups having 1 to 10 carbon atoms, or represent an individual hydrogen atom. Especially preferred is 2,4-bis{[4-(2-ethyl-hexyloxy)-2-hydroxy]phenyl}-6-(4-methoxyphenyl)-1,3,5-triazine (INCl: Aniso triazine), which is available from CIBA-Chemikalien GmbH under the trade name Tinosorb® S.

Especially advantageous preparations for the purposes of the present invention, which are characterized by a high or very high UV-A protection, preferably comprise a plurality of UV-A- and broadband filters, in particular dibenzoylmethane derivatives [for example, 4-(tert.-butyl)-4′-methoxydibenzoylmethane], benzotriazole derivatives [for example, 2,2′-methylene-bis-(6-(2H-benzotriazole-2-yl)-4-(1,1,3,3-tetramethylbutyl)-phenol)], phenylene-1,4-bis(2-benzimidazyl)-3,3′-5,5′-tetrasulfonic acid and/or its salts, 1,4-d(2-oxo-10-sulfo-3-bornylidenemethyl)-benzene and/or the salts thereof, and/or 2,4-bis{[4-(2-ethyl-hexyloxy)-2-hydroxy]phenyl}-6-(4-methoxyphenyl)-1,3,5-triazine, each individually or in any combinations with one another.

Likewise other UV filter substances, which have the structural motif 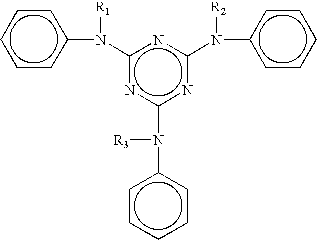
are advantageous filter substances for the purposes of the present invention, for example, s-triazine derivatives as disclosed in EP 570 838 A1, whose chemical structure is represented by the generic formula 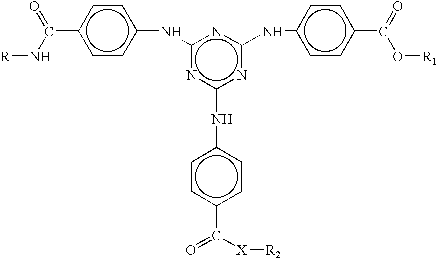
where
R is a branched or unbranched C₁₋₁₈ alkyl residue, a C₅₋₁₂ cycloalkyl residue, if need be, substituted with one of more C₁₋₄ alkyl groups,
X is an oxygen atom or an NH group, and
R₁ is a branched or unbranched C₁₋₁₈ alkyl residue, a C₅₋₁₂ cycloalkyl residue, if need be, substituted with one of more C₁₋₄ alkyl groups, or a hydrogen atom, an alkaline metal, an ammonium group, or a a group of the formula 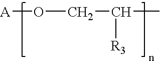
where
   A is a branched or unbranched C₁₋₁₈ alkyl residue, a C₅₋₁₂ cycloalkyl or aryl residue, if need be, substituted with one of more C₁₋₄ alkyl groups,
   R₃ is a hydrogen atom or a methyl group, and
   n is a number from 1 to 10,
R₂ is a branched or unbranched C₁₋₁₈ alkyl residue, a C₅₋₁₂ cycloalkyl residue, if need be, substituted with one of more C₁₋₄ alkyl groups, when X is the NH group, and
a branched or unbranched C₁₋₁₈ alkyl residue, a C₅₋₁₂ cycloalkyl residue, if need be, substituted with one of more C₁₋₄ alkyl groups, or a hydrogen atom, an alkaline metal atom, an ammonium group or a group of the formula 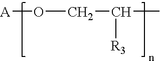
where
   A is a branched or unbranched C₁₋₁₈ alkyl residue, a C₅₋₁₂ cycloalkyl residue, if need be, substituted with one of more C₁₋₄ alkyl groups,
   R₃ is a hydrogen atom or a methyl group, and
   n is a number from 1 to 10,
   when X is an oxygen atom.

Furthermore, a very preferred UV filter substance for the purposes of the present invention is an asymmetrically substituted s-triazine, whose chemical structure is represented by the formula 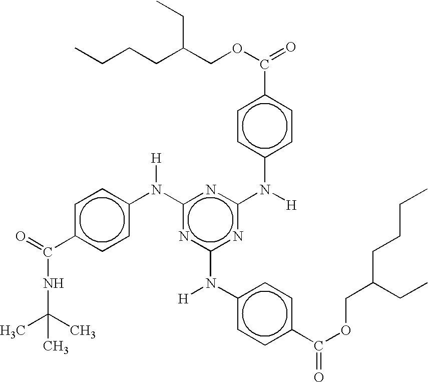
which is in the following also referred to as dioctylbutylamidotriazone (INCl: Dioctyl butamido triazone) and can be obtained from Sigma 3V under the trade name UVASORB HEB.

Advantageous for the purposes of the present invention is also a symmetrically substituted s-triazine, i.e., 4,4′,4″-(1,3,5-triazine-2,4,6-triyltriimino)-tris-benzoic acid-tris(2-ethylhexylester), synonym: 2,4,6-tris-[anilino-(p-carbo-2′-ethyl-1′-hexyloxy)]-1,3,5-triazine (INCl: Octyl Triazone), which BASF Aktiengesellschaft markets under its trade name UVINUL® T 150.

Likewise EP 775 698 discloses bis-resorcinyl triazine derivatives that are to be used with preference and whose chemical structure is represented by the generic formula 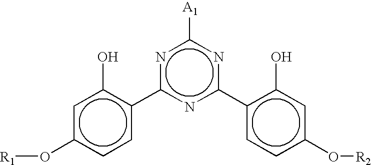
where R₁, R₂, and A₁ represent a great variety of organic residues.

Also advantageous for the purposes of the present invention are 2,4-bis-{[4-(3-sulfonato)-2-hydroxy-propyloxy)-2-hydroxy]-phenyl}-6-(4-methoxyphenyl)-1,3,5-triazine sodium salt, 2,4-bis-{[4-(3-(2-propyloxy)-2-hydroxy-propyloxy)-2-hydroxy]-phenyl}-6-(4-methoxyphenyl)-1,3,5-triazine, 2,4-bis-{[4-(2-ethyl-hexyloxy)-2-hydroxy]-phenyl}-6-[4-(2-methoxyethyl-carboxyl)-phenylamino]-1,3,5-triazine, 2,4-Bis-{[4-(3-(2-propyloxy)-2-hydroxy-propyloxy)-2-hydroxy]-phenyl}-6-[4-(2-ethyl-carboxyl)-phenylamino]-1,3,5-triazine, 2,4-bis-{[4-(2-ethyl-hexyloxy)-2-hydroxy]-phenyl}-6-(1-methyl-pyrrol-2-yl)-1,3,5-triazine, 2,4-bis-{[4-tris(trimethylsiloxy-silylpropyloxy)-2-hydroxy]-phenyl}-6-(4-methoxyphenyl)-1,3,5-triazine, 2,4-bis-{[4-(2″-methylpropenyl-oxy)-2-hydroxy]-phenyl}-6-(4-methoxyphenyl)-1,3,5-triazine, and 2,4-bis-{[4-(1′,1′,1′,3′,5′,5′,5′-heptamethylsiloxy-2″-methyl-propyloxy)-2-hydroxy]-phenyl}-6-(4-methoxyphenyl)-1,3,5-triazine.

An advantageous broadband filter for the purposes of the present invention is 2,2′-methylene-bis-(6-(2H-benzotriazole-2-yl)-4-(1,1,3,3-tetramethylbutyl)-phenol), which is available from CIBA-Chemikalien GmbH under the trade name Tinosorb® M.

A further advantageous broadband filter for the purposes of the present invention is 2-(2H-benzotriazole-2-yl)-4-methyl-6-[2-methyl-3-[1,3,3,3-tetramethyl-1-[(trimethylsilyl)oxy]disiloxanyl]propyl]-phenol (CAS-Nr.: 155633-54-8) under the INCl name Drometrizole Trisiloxane.

The UV-B and/or broadband filters may be oil-soluble or water-soluble. Advantageous oil-soluble UV-B filter and/or broadband filter substances are, for example:
derivatives of 3-benzylidene camphor, preferably 3-(4-methylbenzylidene) camphor, 3-benzylidene camphor;
derivatives of 4-aminobenzoic acid, preferably 2-(ethylhexyl)4-dimethylamino-benzoate, amyl 4-(dimethylamino)-benzoate;
2,4,6-trianilino-(p-carbo-2′-ethyl-1′-hexyloxy)-1,3,5-triazine;
esters of benzalmalonic acid, preferably di(2-ethylhexyl) 4-methoxybenzalmalonate;
esters of cinnamic acid, preferably (2-ethylhexyl)4-methoxy cinnamate, isopentyl 4-methoxy cinnamate;
derivatives of benzophenone, preferably 2-hydroxy-4-methoxybenzophenone, 2-hydroxy-4-methoxy-4′-methylbenzophenone, 2,2′-dihydroxy-4-methoxybenzophenone; as well as
polymer-linked UV filters.

Advantageous water-soluble UV-B and/or broadband filters are, for example:
salts of 2-phenylbenzimidazole-5-sulfonic acid, such as its sodium-, potassium-, or its triethanol ammonium salt, as well as the sulfonic acid itself; and
sulfonic acid derivatives of 3-benzylidene camphor, such as, for example, 4-(2-oxo-3-bornylidene-methyl)benzenesulfonic acid, 2-methyl-5-(2-oxo-3-bornylidenemethyl)sulfonic acid and their salts.

For the purposes of the present invention, especially advantageous, liquid UV filter substances that can be used at room temperature are homomenthylsalicylate (INCl: homosalate), 2-ethylhexyl-2-hydroxybenzoate (2-ethylhexylsalicylate, octylsalicylate, INCl: octyl salicylate), 4-isopropylbenzylsalicylate und esters of the cinnamic acid, preferably 4-meth-oxy cinnamic acid(2-ethylhexyl)ester (2-ethylhexyl-4-methoxycinnamate, INCl: octyl methoxycinnamate), and 4-methoxycinnamic acid-isopentylester (isopentyl-4-methoxycinnamate, INCl: isoamyl p-methoxycinnamate), 3-(4-(2,2-bis-ethoxycarbonylvinyl)-phenoxy)propenyl)-methoxysiloxane/dimethylsiloxane copolymer (INCl: dimethicodiethylbenzalmalonate), which is available, for example, under the trade name Parsol® SLX from Hoffmann La Roche.

A further light protective filter substance that is to be advantageously used in accordance with the invention, is ethylhexyl-2-cyano-3,3-diphenylacrylate (octocrylene), which is available from BASF under the name Uvinul® N539.

It can also be of considerable advantage to use polymer-linked and polymeric UV filter substances in preparations according to the present invention, in particular such as are disclosed in WO-A-92/20690.

The list of the specified UV filters that can be used for the purposes of the present invention, is naturally not intended to be limiting.

Advantageously, the preparations of the invention contain the substances, which absorb UV radiation in the UV-A and/or UV-B range, where the total amount of the filter substances is, for example, from 0.1 wt. % to 30 wt. %, preferably 0.5 wt. % to 20 wt. %, in particular 1.0 to 15.0 wt. % based on the total weight of the preparations, for the purpose of making available cosmetic preparations, which protect hair or skin against the entire range of the ultraviolet radiation. They can also serve as sunscreens for the hair or skin.

Vitamins and Vitamin Derivatives

Among those are the vitamins A, B₁₋₆, B₁₂, C, D, E, F, H, K, and PP, as well as their derivatives. According to the invention, they may be advantageously contained in a concentration from 0.001 to 10 wt. %, preferably 0.05 to 7 wt. %, more preferably 0.5 to 5 wt. % based on the total weight of the preparation.

As preferred vitamin derivates in accordance with the invention retinyl palmitate, ascorbyl glucoside, tocopheryl acetate, tocopheryl palmitate, niacinamide, and panthenol are used.

In accordance with the invention, it is advantageous to use vitamins and their derivatives in a concentration from 0.05 to 1 wt. %, preferably in a concentration from 0.01 to 0.5 wt. %, and more preferably in a concentration from 0.05 to 0.2 wt. % based on the total weight of the preparation.

Enzymes

The cosmetic of the invention or the dispenser that is advantageously used in accordance with the invention advantageously contains enzymes. As enzymes, it is possible and advantageous to use for the purposes of the present invention lipases, esterases, proteases, and other hydrolases. According to the invention, lipases are preferred.

As enzymes in accordance with the invention, it is possible and advantageous to use enzyme extracts from Alcaligenes sp. (for example, from the manufacturer Amano Enzyme Europe Ltd.), Asperillus niger (for example, from the manufacturers Amano Enzyme Europe Ltd. or Fluka), Candida cylindracea (for example, from the manufacturers Sigma, Aldrich, Fluka, Amano Enzyme Europe Ltd., Boeringer-Mannheim), Candida lipolytica (for example, from the manufacturers Amano Enzyme Europe Ltd., Fluka), Chromobacterium viscosium (for example, from the manufacturer Sigma), Humicola laguninosa (for example, from the manufacturer Amano Enzyme Europe Ltd.), Mucor miehei (for example, from the manufacturers Amano Enzyme Europe Ltd., Novo), Penecillum roqueforti (for example, from the manufacturer Fluka), Porcine pancreas (for example, from the manufacturers Sigma, Aldrich, Fluka, Amano Enzyme Europe Ltd., Boeringer-Mannheim), Pseudomonas aegruginosa (for example, from the manufacturer Amano Enzyme Europe Ltd.), Pseudomonas fluorescens (for example, from the manufacturers Amano Enzyme Europe Ltd., Fluka), Pseudomonas sp. (for example, from the manufacturers Sigma, Boeringer-Mannheim), Rhizopus delemar (for example, from the manufacturers Sigma, Fluka, Boeringer-Mannheim), Rhizopusjaponicus (for example, from the manufacturer Amano Enzyme Europe Ltd.), Rhizopus oryzae (for example, from the manufacturer Amano Enzyme Europe Ltd.), Rhizopus sp. (for example, from the manufacturers Amano Enzyme Europe Ltd., Serva), and/or wheat germ (for example, from the manufacturer Sigma).

In this connection, it is preferred in accordance with the invention to use as enzyme extracts lipases from Porcine pancreas, Aspergillus niger, Chromobacterium viscosium, Geotrichum candidum, Penecillum camberti, Penecillum roqueforti.

Quite especially preferred are in accordance with the invention lipases of the Mucor miehei species (for example, Lipozyme® from Novo Nordisk), Humicola laguninosa, Pseudomonas aegruginosa, Pseudomonas fluorescens, Pseudomonas sp., Candida cylindracea, and/or Candida lipolytica.

In accordance with the invention, it is advantageous to have present one or more enzymes in the form of an aqueous solution. Advantageously, the solution contains in accordance with the invention one or more enzymes in a range from 0.1 to 5 wt. %, preferably 1 to 5 wt. %, and more preferably 1 to 2 wt. % based on the total weight of the preparation.

Antioxidants

Advantageously, antioxidants are selected from the group consisting of amino acids (for example, glycine, lysine, arginine, cysteine, histidine, tyrosine, tryptophan) and derivatives thereof (as salt-, ester-, ether-, sugar-, nucleotide-, nucleoside-, peptide-, and lipid compounds); imidazoles (for example, urocanic acid) and their derivatives (as salt-, ester-, ether-, sugar-, nucleotide-, nucleoside-, peptide-, and lipid compounds); peptides, such as D,L-carnosine, D-carnosine, L-carnosine, anserine, and derivatives thereof (for example, as salt-, ester-, ether-, sugar-, nucleotide-, nucleoside-, peptide-, and lipid compounds); carotenoids, carotene (for example, α-carotene, β-carotene, ψ-lycopene, phytoene) and derivatives thereof (for example, as salt-, ester-, ether-, sugar-, nucleotide-, nucleoside-, peptide-, and lipid compounds); chlorogenic acid and its derivates (as salt-, ester-, ether-, sugar-, nucleotide-, nucleoside-, peptide-, and lipid compounds); aurothioglucose, propylethiouracil and other thiols (for example, thioredoxin, lipoic acid, glutathione, cysteine, cystine, cystamine, and glycosyl-, N-acetyl-, methyl-, ethyl-, propyl-, amyl-, butyl- and lauryl-, palmitoyl-, oleyl-, □-linoleyl-, cholesteryl-, and glyceryl esters thereof, as well as their salts, dilauryl thiodipropionate, distearyl thiodipropionate, thiodipropionic acid and derivatives thereof (as salt-, ester-, ether-, sugar-, thiol-, nucleotide-, nucleoside-, peptide and/or lipid compounds), and sulfoximine compounds (for example, homocysteine sulfoximine, buthionine sulfones, penta-, hexa-, heptathionine sulfoximine) in very low tolerated doses (for example, pmol to □mol/kg). Furthermore, metal chelating agents (for example, Apoferritin, Desferral, lactoferrin, □-hydroxy fatty acids, palmitic acid, phytic acid), and derivatives thereof (as salt-, ester-, ether-, sugar-, thiol-, nucleotide-, nucleoside-, peptide and/or lipid compounds); □-hydroxy acids (for example, citric acid, lactic acid, malic acid), humic acid, bile acid, bile extracts, bilirubin, biliverdin, EDTA, EGTA and derivatives thereof; unsaturated fatty acids and derivatives thereof; unsaturated fatty acids and their derivatives (for example, □-linolenic acid, linoleic acid, oleic acid), folic acid, and derivatives thereof; furfurylidene sorbitol and its derivatives; ubiquinone, ubiquinol, plastoquinone and their derivatives (as salt-, ester-, ether-, sugar-, thiol-, nucleotide-, nucleoside-, peptide and lipid compounds); vitamin C and derivatives (for example, ascorbyl palmitate, Mg-ascorbyl phosphate, ascorbyl acetate); tocopherols and derivatives thereof (for example, vitamin E acetate) as well as phenolic compounds and plant extracts containing same, such as, for example, flavonoids (for example, glycosylrutin, ferulic acid, caffeic acid); furfurylidene glucitol, butylhydroxytoluene, butylhydroxyanisole, nordihydroguaiac resin acid; nordihydroguaiaretic acid; trihydroxybutyrophenone and derivatives thereof (as salt-, ester-, ether-, sugar-, nucleotide-, nucleoside-, peptide and lipid compounds); uric acid and its derivatives; mannose and its derivatives (as salt-, ester-, ether-, sugar-, nucleotide-, nucleoside-, peptide and lipid compounds); zinc and its derivatives (for example, ZnO, ZnSO₄); selenium and its derivatives (for example, selenium methionine, Ebselen); stilbene and its derivatives for example, stilbene oxide, trans-stilbene oxide); and derivatives (salts, esters, ethers, sugar, nucleotides, nucleosides, peptides, and lipids) of these referenced active ingredients, which are suitable according to the invention.

The amount of the foregoing antioxidants (one or more compounds) in the preparations is preferably from 0.001 to 30 wt. %, more preferably 0.05 to 20 wt. %, in particular 1 to 10 wt. % based on the total weight of the preparation.

If vitamin E and/or its derivatives is or are the antioxidant or antioxidants, it will be advantageous to choose their respective concentration from the range of 0.001 to 10 wt. % based on the total weight of the formulation.

If vitamin A, or vitamin-A derivatives, or carotenes, or their derivatives is or are the antioxidant or antioxidants, it will be advantageous to choose the respective concentrations from the range of 0.001 to 10 wt. % based on the total weight of the formulation.

Antiwrinkle Agents, Plant Extracts, and Other Active Ingredients

Further advantageous active ingredients for the purpose of the present invention are natural ingredients and/or their derivatives, such as, for example, alpha-lipoic acid, phytoene, D-biotin, coenzyme Q10, alpha-glucosylrutin, carnitine, carnosine, natural and/or synthetic isoflavonoids, creatine, creatinine, lignans, taurine, and/or beta-alanine.

According to the invention, however, it is also possible and advantageous to incorporate other pharmaceutically or dermatologically active substances, such as, for example, skin-soothing and skin-caring substances. Among those are, for example, panthenol, allantoin, tannin, as well as plant ingredients, such as azulen and bisabolol, glycyrrhizin, hamamelin, and plant extracts, such as chamomile, aloe vera, hamamelis, and the root of licorice.

The concentration of the foregoing active ingredient (one or more compounds) in the preparations is preferably 0.001 to 30 wt. %, more preferably 0.05 to 20 wt. %, in particular 1-10. wt. % based on the total weight of the preparation.

Selftanners

As selftanners, it is advantageous in accordance with the invention to use, inter alia:
glycerolaldehyde, hydroxymethylglyoxal, gamma-dialdehyde, erythrulose, 6-aldo-D-fructose, ninhydryn, 5-hydroxy-1,4-naphtoquinone (Juglon), 2-hydroxy-1,4-napthoquinone (Lawson).

Especially preferred for the purposes of the present invention is 1,3-dihydroxyacetone (DHA). Of advantage is a concentration of 1,3-dihydroxyacetone from 0.5 to 10 wt. %, in particular a concentration from 1 to 7 wt. % based on the total weight of the preparation.

Depigmentors

As advantageous depigmentors, it is possible to use in accordance with the invention, for example, dicarboxylic acids, such as 8-hexadecene-1,16-dicarboxylic acid (dioic acid, CAS Number 20701-68-2), kojic acid, ascorbic acid, and azelaic acid, as well as their derivatives.

Advantageously, the total concentration of depigmentors in the corresponding preparation is in accordance with the invention in a range from 0.001 to 10 wt. %, preferably 0.0005 to 8 wt. %, in particular 0.05 to 5 wt. %.

Repellents

According to the invention, it is possible and advantageous to use:
N,N-diethyl-3-methylbenzamide (DEET), dimthylphthalate (Palatinol M, DMP), 2,3,4,5,-bis-(2-butylene)-tetrahydro-2-furaldehyde (MGK-Repellent 11), butopyronoxyl (Indalone), N,N-caprylic acid diethylamide (Repellent 790), o-chloro-N,N-diethyl benzamide in mixture with N,N-diethylbenzamide (Kik Repellent), diemthylcarbate (Dimalone), di-n-propylisocinchomeronate (MGK Repellent 326), 2-ethylhexane-1,3-diol (Rutgers 612), n-octyl-bicycloheptene dicarboximide (MGK 264 insecticide synergist), piperonyl-butoxide (PBO), and very preferred by the invention the ester of 3-(N-n-butyl-N-acetyl-amino)propionic acid (also named Repellent 3535), as well as the 1-piperidinecarboxylic acid-2-(2-hydroxyethyl)-1-methylpropyl ester (Bayrepel®).

In accordance with the invention, it is advantageous to use a content of repellents from 0.005 to 70.0 wt. %, in particular 0.01 to 50.0 wt. %, and most preferably 3 to 15 wt. % based on the total weight of the preparation.

Waterfree Polyols

In accordance with the invention, it is preferred to select the polyalcohols from the group of the substances with a molecular weight from 200 to 600 g/mol, for example, polyethyleneglycol (200) (=PEG-4), polyethyleneglycol (300) (=PEG-6), polyethyleneglycol (400) (=PEG-8), polyethyleneglycol (1000) (=PEG-5), and polyethyleneglycol (1200) (=PEG-6).

Especially preferred polyalcohols in accordance with the invention are the compounds polyethyleneglycol (400) (=PEG-8), polyethyleneglycol (1000) (=PEG-5), and polyethyleneglycol (1200) (=PEG-6).

In accordance with the invention a polyalcohol concentration from 5 to 90 wt. % is preferred. Especially preferred is a concentration from 40 to 85 wt. % based on the total weight of the preparation.

In accordance with the invention, “waterfree” are those polyols, whose water concentration is smaller than 5 wt. %, and preferably smaller than 3 wt. % based on the total weight of the polyols.

According to the invention, polyol-containing preparations are advantageously offered in particular as almost waterfree preparations, so that when they are applied and in contact with water, they can be applied as so-called “self-warming” cosmetic or dermatic compositions (because they release heat upon contact with water).

Coloring and Effect Materials

As pigment dyes that are advantageous according to the invention, it is possible to use all listed compounds from the corresponding positive list of the Cosmetic Directive or the EC list of cosmetic colorants. In most cases they are identical with the dyes approved for foods. Besides color pigments, the preparations of the invention can also contain additional dyes. Advantageous color pigments are, for example, titanium dioxide, mica, iron oxides (for example, Fe₂O₃, Fe₃O₄, FeO(OH)) and/or tin oxide. Advantageous dyes are, for example, carmine, Berlin blue, chromic oxide green, ultramarine blue, and/or manganese violet. It is particularly advantageous to choose the dyes and/or color pigments from the following list. The color index numbers (CIN) are taken from the Rowe Colour Index, 3^{rd} edition, Society of Dyers and Colourists, Bradford, England, 1971.

| CHEMICAL OR OTHER NAME | CIN | Color |
|---|---|---|
| Pigment Green | 10006 | green |
| Acid Green 1 | 10020 | green |
| 2,4-Dinitrohydroxynaphthalene-7-sulfonic acid | 10316 | yellow |
| Pigment Yellow 1 | 11680 | yellow |
| Pigment Yellow 3 | 11710 | yellow |
| Pigment Orange 1 | 11725 | orange |
| 2,4-Dihydroxyazobenzene | 11920 | orange |
| Solvent Red 3 | 12010 | red |
| 1-(2′-Chloro-4′-nitro-1′-phenylazo)-2- | 12085 | red |
| hydroxynaphthalene | | |
| Pigment Red 3 | 12120 | red |
| Ceres red; Sudan red; Fat Red G | 12150 | red |
| Pigment Red 112 | 12370 | red |
| Pigment Red 7 | 12420 | red |
| Pigment Brown 1 | 12480 | brown |
| 4-(2′-Methoxy-5′-sulfodiethylamido-1′-phenylazo)- | 12490 | red |
| 3-hydroxy-5″-chloro-2″,4″-dimethoxy-2- | | |
| naphthanilide | | |
| Disperse Yellow 16 | 12700 | yellow |
| 1-(4-Sulfo-1-phenylazo)-4-aminobenzene-5-sulfonic | 13015 | yellow |
| acid | | |
| 2,4-Dihydroxyazobenzene-4′-sulfonic acid | 14270 | orange |
| 2-(2,4-Dimethylphenylazo-5-sulfo)-1-hydroxy- | 14700 | red |
| naphthalene-4-sulfonic acid | | |
| 2-(4-Sulfo-1-naphthylazo)-1-naphthol-4-sulfonic | 14720 | red |
| acid | | |
| 2-(6-Sulfo-2,4-xylylazo)-1-naphthol-5-sulfonic | 14815 | red |
| acid | | |
| 1-(4′-Sulfophenylazo)-2-hydroxynaphthalene | 15510 | orange |
| 1-(2-Sulfo-4-chloro-5-carboxy-1-phenylazo)-2- | 15525 | red |
| hydroxynaphthalene | | |
| 1-(3-Methylphenylazo-4-sulfo)-2-hydroxy- | 15580 | red |
| naphthalene | | |
| 1-(4′,(8′)-Sulfonaphthylazo)-2-hydroxy- | 15620 | red |
| naphthalene | | |
| 2-Hydroxy-1,2′-azonaphthalene-1′-sulfonic acid | 15630 | red |
| 3-Hydroxy-4-phenylazo-2-naphthylcarboxylic acid | 15800 | red |
| 1-(2-Sulfo-4-methyl-1-phenylazo)-2-naphthylcar- | 15850 | red |
| boxylic acid | | |
| 1-(2-Sulfo-4-methyl-5-chloro-1-phenylazo)-2- | 15865 | red |
| hydroxynaphthalene- 3-carboxylic acid | | |
| 1-(2-Sulfo-1-naphthylazo)-2-hydroxynaphthalene-3- | 15880 | red |
| carboxylic acid | | |
| 1-(3-Sulfo-1-phenylazo)-2-naphthol-6-sulfonic | 15980 | orange |
| acid | | |
| 1-(4-Sulfo-1-phenylazo)-2-naphthol-6-sulfonic | 15985 | yellow |
| acid | | |
| Allura Red | 16035 | red |
| 1-(4-Sulfo-1-naphthylazo)-2-naphthol-3,6- | 16185 | red |
| disulfonic acid | | |
| Acid Orange 10 | 16230 | orange |
| 1-(4-Sulfo-1-naphthylazo)-2-naphthol-6,8- | 16255 | red |
| disulfonic acid | | |
| 1-(4-Sulfo-1-naphthylazo)-2-naphthol-3,6,8- | 16290 | red |
| trisulfonic acid | | |
| 8-Amino-2-phenylazo-1-naphthol-3,6-disulfonic | 17200 | red |
| acid | | |
| Acid Red 1 | 18050 | red |
| Acid Red 155 | 18130 | red |
| Acid Yellow 121 | 18690 | yellow |
| Acid Red 180 | 18736 | red |
| Acid Yellow 11 | 18820 | yellow |
| Acid Yellow 17 | 18965 | yellow |
| 4-(4-Sulfo-1-phenylazo)-1-(4-sulfophenyl)-5- | 19140 | yellow |
| hydroxy-pyrazolone-3-carboxylic acid | | |
| Pigment Yellow 16 | 20040 | yellow |
| 2,6-(4′-Sulfo-2″,4″-dimethyl)bisphenylazo)-1,3- | 20170 | orange |
| dihydroxybenzene | | |
| Acid Black 1 | 20470 | black |
| Pigment Yellow 13 | 21100 | yellow |
| Pigment Yellow 83 | 21108 | yellow |
| Solvent Yellow | 21230 | yellow |
| Acid Red 163 | 24790 | red |
| Acid Red 73 | 27290 | red |
| 2-[4′-(4″-Sulfo-1″-phenylazo)-7′-sulfo-1′- | 27755 | black |
| naphthylazo]-1-hydroxy-7-aminonaphthalene-3,6- | | |
| disulfonic acid | | |
| 4′-[(4″-Sulfo-1″-phenylazo)-7′-sulfo-1′- | 28440 | black |
| naphthylazo]-1-hydroxy-8-acetylaminonaphthalene- | | |
| 3,5-disulfonic acid | | |
| Direct Orange 34, 39, 44, 46, 60 | 40215 | orange |
| Food Yellow | 40800 | orange |
| trans-β-Apo-8′-carotenaldehyde (C₃₀) | 40820 | orange |
| trans-Apo-8′-carotenoic (C₃₀)-ethyl ester | 40825 | orange |
| Canthaxanthin | 40850 | orange |
| Acid Blue 1 | 42045 | blue |
| 2,4-Disulfo-5-hydroxy-4′-4″-bis(diethyl- | 42051 | blue |
| amino)triphenylcarbinol | | |
| 4-[(4-N-Ethyl-p-sulfobenzylamino)phenyl(4- | 42053 | green |
| hydroxy-2-sulfophenyl)(methylene)-1-(N-ethyl-N- | | |
| p-sulfobenzyl)-2,5-cyclohexadienimine] | | |
| Acid Blue 7 | 42080 | blue |
| (N-Ethyl-p-sulfobenzylamino)phenyl(2-sulfo- | 42090 | blue |
| phenyl)methylene-(N-ethyl-N-p-sulfoben- | | |
| zyl)Δ^{2,5}-cyclohexadienimine | | |
| Acid Green 9 | 42100 | green |
| Diethyldisulfobenzyl-di-4-amino-2-chloro-di-2- | 42170 | green |
| methyl-fuchsonimmonium | | |
| Basic Violet 14 | 42510 | violet |
| Basic Violet 2 | 42520 | violet |
| 2′-Methyl-4′-(N-ethyl-N-m-sulfobenzyl)amino-4″- | 42735 | blue |
| (N-diethyl)amino-2-methyl-N-ethyl-N-m- | | |
| sulfobenzylfuchsonimmonium | | |
| 4′-(N-Dimethyl)amino-4″-(N-phenyl)aminonaphtho- | 44045 | blue |
| N-dimethyl-fuchsonimmonium | | |
| 2-Hydroxy-3,6-disulfo-4,4′-bisdimethylamino- | 44090 | green |
| naphtho-fuchsonimmonium | | |
| Acid Red 52 | 45100 | red |
| 3-(2′-Methylphenylamino)-6-(2′-methyl-4′-sulfo- | 45190 | violet |
| phenylamino)-9-(2″-carboxyphenyl)xanthenium salt | | |
| Acid Red 50 | 45220 | red |
| Phenyl-2-oxyfluorone-2-carboxylic acid | 45350 | yellow |
| 4,5-Dibromofluorescein | 45370 | orange |
| 2,4,5,7-Tetrabromofluorescein | 45380 | red |
| Solvent Dye | 45396 | orange |
| Acid Red 98 | 45405 | red |
| 3′,4′,5′,6′-Tetrachloro-2,4,5,7-tetrabromo- | 45410 | red |
| fluorescein | | |
| 4,5-Diiodofluorescein | 45425 | red |
| 2,4,5,7-Tetraiodofluorescein | 45430 | red |
| Quinophthalone | 47000 | yellow |
| Quinophthalonedisulfonic acid | 47005 | yellow |

It may also be favorable to choose as the dye one or more substances from the following groups: 2,4-dihydroxyazobenzene, 1-(2′-chloro-4′-nitro-1′-phenylazo)-2-hydroxynaphthalene, Ceres Red, 2-(4-sulfo-1-naphthylazo)-1-naphthol-4-sulfonic acid, calcium salt of 2-hydroxy-1,2′azonaphthalene-1′-sulfonic acid, calcium and barium salts of 1-(2-sulfo-4-methyl-1-phenylazo)-2-naphthylcarboxylic acid, calcium salt of 1-(2-sulfo-1-naphthylazo)-2-hydroxynaphthalene-3-carboxylic acid, aluminum salt of 1-(4-sulfo-1-phenylazo)-2-naphthol-6-sulfonic acid, aluminum salt of 1-(4-sulfo-1-naphthylazo)-2-naphthol-3,6-disulfonic acid, 1-(4-sulfo-1-naphthylazo)-2-naphthol-6,8-disulfonic acid, aluminum salt of 8-amino-2-phenylazo-1-napthol-3,6-disulfonic acid, aluminum salt of 4-(4-sulfo-1-phenylazo)-1-(4-sulfophenyl)-5-hydroxypyrazolone-3-carboxylic acid, 4′-[(4″-sulfo-1″-phenylazo-7′-sulfo-1′naphtylazo)-1-hydroxy-8-acetylaminonaphtalene-3,5-disulfonic acid, aluminum and zirconium salts of 4,5-dibromofluorescein, aluminum and zirconium salts of 2,4,5,7-tetrabromofluorescein, 3′,4′,5′,6′-tetrachloro-2,4,5,7-tetrabromofluorescein and its aluminum salt, aluminum salt of 2,4,5,7-tetraiodofluorescein, aluminum salt of quinophthalone disulfonic acid, aluminum salt of indigo disulfonic acid, 4,4′-dimethyl-6,6′-dichlorothioindigo, complex salt (Na, Al, Ca) of carminic acid, red and black iron oxide (CIN: 77491 (red) and 77 499 (black)), hydrated iron oxide (CIN: 77492), manganese ammonium diphosphate (CIN 77745), ultramarine (CIN 77007), and titanium dioxide.

Titanium dioxides of the invention, which may be present in the crystalline modification rutile as well as anatase are advantageously surface treated (“coated”) for the purposes of the present invention. In this process, it is intended to form or maintain, for example, a hydrophilic, amphiphilic, or hydrophobic character. This surface treatment may consist in that the pigments are provided by methods known per se with a thin hydrophilic and/or hydrophobic, inorganic and/or organic coating. For the purposes of the present invention, the different surface coatings may also contain water.

Inorganic surface coatings for the purposes of the present invention may consist of aluminum dioxide (Al₂O₃), aluminum hydroxide Al(OH)₃ or aluminum oxide hydrate (also: alumina, CAS No. 1333-84-2), sodium hexametaphoshpate (NaPO₃)₆. sodium metaphosphate (NaPO₃)ₙ, silicon dioxide (SiO₂) (also: silica, CAS No. 7631-86-9), zirconium oxide (ZrO₂), or iron oxide (FE₂O₃). These inorganic surface coatings may be present alone, or in combination, and/or in combination with organic coating materials.

To this end, oxides, oxide hydrates, or phosphates, for example, of the elements Al, Si, Zr are precipitated in thick layers to the pigment surface.

The inorganic aftertreatment generally occurs in an aqueous suspension of the pigment by adding soluble aftertreatment chemicals, such as, for example, aluminum sulfate, and by subsequently precipitating the slightly soluble hydroxide in the neutral range by a purposeful adjustment of the pH value with sodium hydroxide solution.

Following the inorganic aftertreatment, the coated pigments are separated from the suspension by filtration, and carefully washed to remove the dissolved salts. Finally, the isolated pigments are dried.

Particularly preferred for the purposes of the present invention are titanium dioxides, whose surface was treated with aluminum hydroxide, such as, for example, the titanium dioxide types C47-051 and C47-5175, which are available from Sun Chemical. Further preferred pigments are titanium dioxides which have been coated with aluminum and/or silicon oxides, such as, for example, those from Krosnos Titan: Kronos 1071 and 1075, or from Kingfisher: A310.03 Tudor Aspen.

Organic surface coatings for the purposes of the present invention may consist of vegetable or animal aluminum stearate, vegetable or animal stearic acid, lauric acid, dimethylpolysiloxane (also: dimethicone), methylpolysiloxanes (methicone), simethicone (a mixture of dimethylpolysiloxane with an average chain length from 200 to 350 dimethylsiloxane units and silica gel), or alginic acid. These organic surface coatings may be present alone, or in combination, and/or in combination with inorganic coating materials.

In accordance with the invention it may further be advantageous to use pearlescent pigments. Among those are natural pearlescent pigments, such as, for example
“pearl essence” (guanine/hypoxanthine mixed crystals from fish scales), and
“mother of pearl” (ground mussel shells),
   monocrystalline pearlescent pigments, such as, for example, bismuth oxychloride (BiOCI), and
   layer-substrate pigments, for example, mica/metal oxide.

Bases for pearlescent pigments are, for example, pulverulent pigments or castor oil dispersions of bismuth oxychloride and/or titanium dioxide, and bismuth oxychloride and/or titanium dioxide on mica. The luster pigment listed under CIN 77163, for example, is particularly advantageous.

Also advantageous are, for example, the following types of pearlescent pigments based on mica/metal oxide:

| | Coating/layer | |
|---|---|---|
| Group | thickness | color |
| Silver-white pearlescent | TiO₂: 40-60 nm | silver |
| pigments | | |
| Interference pigments | TiO₂: 60-80 nm | yellow |
| | TiO₂: 80-100 nm | red |
| | TiO₂: 100-140 nm | blue |
| | TiO₂: 120-160 nm | green |
| Color luster pigments | Fe₂O₃ | bronze |
| | Fe₂O₃ | copper |
| | Fe₂O₃ | red |
| | Fe₂O₃ | red- |
| | | violet |
| | Fe₂O₃ | red- |
| | | green |
| | Fe₂O₃ | black |
| Combination pigments | TiO₂/Fe₂O₃ | gold |
| | | shades |
| | TiO₂/Cr₂O₃ | green |
| | TiO₂/Berlin blue | deep |
| | | blue |
| | TiO₂/Carmine | red |

Particularly preferred are the pearlescent pigments that are available from Merck under the trade names Timiron, Colorona, or Dichrona.

Naturally, the list of the referenced pearlescent pigments is not intended to be limiting. Pearlescent pigments which are advantageous for the purposes of the present invention are obtainable by numerous methods known per se. For example, other substrates apart from mica can also be coated with further metal oxides, such as, for example, silica and more of the like. Advantageous are, for example, SiO₂ particles coated with TiO₂ and Fe₂O₃ (“Ronaspheres”), which are marketed by Merck.

It can moreover be also advantageous to do completely without a substrate, such as mica. Particularly preferred are pearlescent pigments, which are prepared with the use of SiO₂. Such pigments, which can also additionally have gonichromatic effects, are available from BASF, for example, under the trade name Sicopearl Fantastico.

In addition, it is possible and advantageous to use pigments from Engelhard/Mearl based on calcium sodium borosilicate coated with titanium dioxide. These are available under the name Reflecks. In addition to the color, they have a glitter effect as a result of their particle size from 40-180 μm.

The dyes and pigments may be present both individually and in a mixture and can be mutually coated with one another, with different coating thicknesses generally producing different color effects.

The particularly preferred pigment dyes in accordance with the invention are the listed blue pigments, such as, for example, INCl: Cl 77007 Outremer Supercosmetique W 6803 from Les Colorant Wackherr, INCl: Cl 77891+mica+silica, Timiron Splendid Blue from Merck.

Also preferred in accordance with the invention are mica coated with titanium dioxide and silicon dioxide, for example, INCl: mica+Cl 77891+silica, Timiron Arctic Silver from Merck, INCl: mica+Cl 77891, Timiron Gleamer Flake MP-45 from Merck; aluminum oxide or silicon dioxide coated with titanium dioxide and tin oxide, for example, INCl: silica+Cl 77891+tin oxide, Xirona Magic Mauve from Merck; for example, INCl: alumina+Cl 77891+tin oxide, Xirona Silver, mica coated with titanium dioxide and Berlin blue, for example, INCl: mica+Cl 77891+Cl 77510, Colorona Light Blue and Colorona Dark Blue from Merck.

Pearl Luster:

It is also preferred to use pearlescent substances based on dialkyl ethers, which are solid at 30° C. and have, for example, the following formula: R—O—R′. R and R′ may be identical or different, straight-chain or branched, saturated or unsaturated alkyl radicals. These may consist of 12 to 30 carbon atoms, preferably 14 to 24 carbon atoms. More preferably, R and R′ consist of a stearyl radical (for example, INCl: distearyl ether, Cutina STE from Cognis).

The dialkyl ethers in use are not water soluble in a concentration greater than 0.1% at 25° C.

It is preferred and possible to use also acylated radicals, which consist of a fatty aid chain with 8 to 30 carbon atoms.

Each acylated derivative contains at least one RC(═O) group, wherein R is a fatty acid chain with 8 to 30 carbon atoms.

Used are in particular ethylene glycol monostearates and ethylene glycol distearates, for example, INCl: glycol distearate, Cutina AGS from Cognis, INCl: Aqua+glycol distearate+glycerol+laureth-4+cocamidopropyl betaine, Euperlan PK 3000 OK from Cognis, INCl: PEG-3 distearate, Cutina TS from Cognis.

These are advantageously used in accordance with the invention in a concentration from 0.5% to 2%.

Opacifiers:

In accordance with the invention, it is also possible and advantageous to use opacifiers. Preferred are the sodium salts of a polymer of styrene with a monomer consisting of acrylic acid, methacrylic acid, or another olefin and one of their esters, for example, INCl: sodium styrene/acrylates copolymer, Acusol OP 301 from Rohm & Haas. These are advantageously used in accordance with invention in a concentration from 0.5% to 2%.

According to the invention, it is also possible and advantageous to include glitter and sparkle particles in at least one of the preparations.

In accordance with the invention, the preparations of the invention may advantageously contain one or more surfactants, which can be anionic, cationic, nonioinic, and zwitterionic.

Advantageous wash-active anionic surfactants for the purposes of the present invention are acylamino acids and their salts, such as
acyl glutamates, in particular sodium acyl glutamate
sarcosinates, for example, myristoyl sarcosinate, TEA-lauroyl sarcosinate, sodium lauroyl sarcosinate, and sodium cocoyl sarcosinate,
   Sufonic acids and salts, such as:
Acyl isethionate, for example, sodium-ammoniumcocoyl isethionate;
Sulfosuccinates, for example, dioctyl sodium sulfosuccinate, disodium laureth sulfosuccinate, disodium lauryl sulfosuccinate, and disodium undecylenamido-MEA-sulfosuccinate, disodium PEG-5 laurylcitrate sulfosuccinate, and derivatives, as well as esters of sulfuric acid, such as:
Alkyl ether sulfonates, for example, sodium-, ammonium-, magnesium-, MIPA-, TIPA-laureth sulfate, sodium myreth sulfate, and sodium C₁₂₋₁₃pareth sulfate; and
Alkyl sulfates, for example, sodium, ammonium, and TEA-lauryl sulfate.

Advantageous wash-active surfactants for the purposes of the present invention are quaternary surfactants. Quaternary surfactants contain at least one N-atom which is covalently bonded to 4 alkyl or aryl groups. Advantageous are, for example, alkylbetaine, alkylamidopropylbetaine, and alkylamidohydroxysultaine.

Advantageous wash-active amphoteric surfactants for the purposes of the present invention are
acyl/dialkylethylenediamine, for example sodium acyl amphoacetate, disodium acyl amphodipropionate, disodium alkyl amphodiacetate, sodium acyl amphohydroxypropylsulfonate, disodium acyl amphodiacetate, and sodium acyl amphopropionate.
   Advantageous wash-active nonionic surfactants for the purposes of the present invention are
alkanolamides, such as cocamides MEA/DEA/MIPA,
esters, which are formed by esterification of carboxylic acids with ethylene oxide, glycerol, sorbitane, or other alcohols,
ether, for example ethoxylated alcohols, ethoxylated lanolin, ethoxylated polysiloxanes, propoxylated POE ethers, and alkylpolyglycosides, such as laurylglucoside, decylglycoside, and cocoglycoside.
   Further advantageous anionic surfactants are
taurates, for example, sodium lauroyl taurate and sodium methylcocoyl taurate,
ether carboxylic acids, for example, sodium laureth-13 carboxylate, and sodium PEG-6 cocoamide carboxylate, sodium PEG-7 olive oil carboxylate,
esters of phosphoric acid and salts, such as, for example, DEA-oleth-10-phosphate and dilaureth-4 phosphate,
alkyl sulfonates, for example, sodium cocomonoglyceride sulfate, sodium C₁₂₋₁₄olefin sulfonate, sodium lauryl sulfoacetate, and magnesium PEG-3 cocamide sulfate.
   Additional advantageous amphoteric surfactants are:
N-alkylamino acids, for example, aminopropylalkyl glutamide, alkylaminopropionic acid, sodium alkylimidodipropionate, and lauroamphocarboxyglycinate, and N-coconut fatty acid-amidoethyl-N-hydroethylglycinate, sodium salts and their derivatives.
   Additional advantageous nonionic surfactants are alcohols.

Further suitable anionic surfactants for the purposes of the present invention also are
acylglutamates, such as Di-TEA-palmitoylaspartate and sodium caprylic/capric glutamate,
acylpeptides, for example palmitoyl-hydrolized milk protein, sodium cocoyl-hydrolized soy protein, and sodium-/potassium cocoyl-hydrolized collagen,
   as well as carboxylic acids and derivatives, such as
for example, lauric acid, aluminum stearate, magnesium alkanolate, and zincundecylenate,
ester-carboxylic acids, for example, calcium stearoyllactylate, laureth-6 citrate, and sodium PEG-4 lauramide carboxylate,
alkylaryl sulfonates.

Further suitable cationic surfactants for the purposes of the present invention also are
alkylamines,
alkylimidazoles,
ethoxylated amines,
in particular their salts.

Further suitable nonionic surfactants for the purposes of the present invention also are amine oxides, such as cocoamidopropylamine oxide.

It is advantageous to select the wash-active surfactant or surfactants of the invention from the group of the surfactants, which have an HLB value greater than 25, particularly advantageous are those, which have an HLB value greater than 35.

Preferred according to the invention are alkyl sulfates or alkyl ether sulfates or a surfactant combination of alkyl ether sulfates with amphoteric or nonionic surfactants. Particularly preferred is a surfactant combination of alkyl ether sulfates with alkylamidopropylbetaines or alkylamphoacetates or alkylpolyglucosides. Particularly preferred are also combinations of alkyl ether sulfates with alkylamidopropylbetaines or alkylamphoacetates and acylglutamates.

It is advantageous for the purposes of the invention to select the content of one or more wash-active surfactants in the corresponding preparation from the range of 1 to 30 wt. %, preferably 5 to 25%, more preferably 10 to 20 wt. % based on the total weight of the preparation.

According to the invention, surfactant-containing preparations may be advantageously used as cleansing preparations. However in accordance with the invention, the content of surfactants in the preparations of the invention can be also advantageously used for foaming the preparations (embodiment: foam dispenser).

In accordance with the invention, the preparations of the invention may advantageously contain polysorbates. Polysorbates are a class of compounds that derive from sorbitan, a furan derivative that is obtained from sorbitol by the separation of two equivalents of water. The hydroxyl groups of the sorbitan are etherified with polyethylene glycols, whose ends may be esterified with fatty acids. They can be represented by the formula 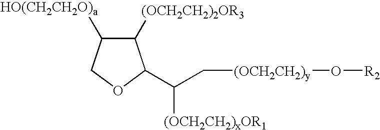
where R₁, R₂, R₃=H, fatty acid residue.

For the purposes of the invention, advantageous polysorbates are, for example,
Polyoxyethylene(20)sorbitanmonolaurate (Tween 20, CAS-No. 9005-64-5)
Polyoxyethylene(4)sorbitanmonolaurate (Tween 21, CAS-No. 9005-64-5)
Polyoxyethylene(4)sorbitanmonostearate (Tween 61, CAS-No. 9005-67-8)
Polyoxyethylene(20)sorbitantristearate (Tween 65, CAS-No. 9005-71-4)
Polyoxyethylene(20)sorbitanmonooleate (Tween 80, CAS-No. 9005-65-6)
Polyoxyethylene(5)sorbitanmonooleate (Tween 81, CAS-No. 9005-65-5)
Polyoxyethylene(20)sorbitantrioleate (Tween 85, CAS-No. 9005-70-3).

According to the invention, these polysorbates are advantageously used, individually or as mixture of several polysorbates, in a concentration from 0.1 to 5 wt. %, and in particular in a concentration of 1.5 to 2.5 wt. % based on the total weight of the preparation.

Hydrocolloids

The preparations of the invention advantageously contain in accordance with the invention one or more hydrocolloids. “Hydrocolloid” is the technological short name for a per se more correct description “hydrophilic colloid.” Hydrocolloids, also named thickeners or gel formers, are macromolecules, which have a largely linear configuration and dispose of intermolecular interactive forces that enable secondary and primary valency bonds between the individual molecules and thus the formation of a netlike structure. They are in part water-soluble, natural or synthetic polymers, which form in aqueous systems gels or viscous solutions. They increase the viscosity of water in that they bind either water molecules (hydration) or, however, that they absorb and encapsulate water into their interconnected macromolecules, while simultaneously limiting the movability of water. Such water-soluble polymers represent a large group of chemically very different, natural and synthetic polymers, whose common characteristic is their solubility in water or aqueous media. A precondition therefore is that these polymers have a number of hydrophilic groups that is adequate for the solubility in water, and that they are not considerably crosslinked. The hydrophilic groups can be of nonioinic, anionic, or cationic nature, for example, as follows:

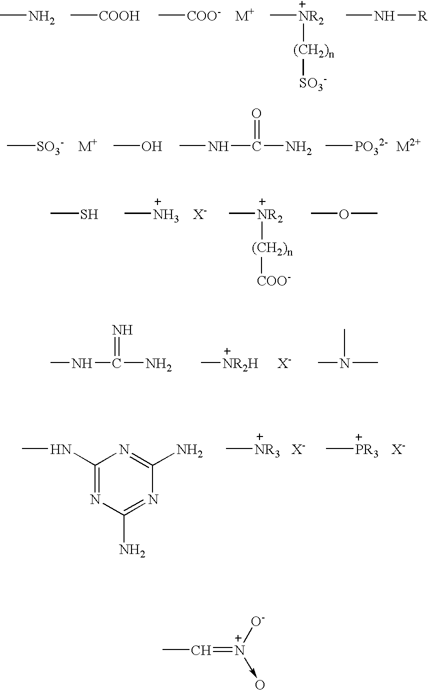

The group of the cosmetically and dermatologically relevant hydrocolloids can be classified as follows:
organic, natural compounds, such as, for example, agar-agar, carrageen, tragacanth, gum arabicum, alginates, pectins, polyoses, guar meal, carob bean meal, starch, dextrines, gelatins, casein, organic, modified natural substances, such as, for example, carboxymethyl cellulose and other cellulose ethers, hydroxyethyl- and hydroxypropyl cellulose, and the like, organic, fully synthetic compounds, such as, for example, polyacryl and polymethacryl compounds, vinyl polymers, polycarboxylic acids, polyethers, polyimines, polyamides, inorganic compounds, such as, for example, polysilicic acids, clay minerals, such as, montmorillonite, zeolithe, silicic acids.

As advantageous hydrocolloids in accordance with the invention, one uses agar-agar, carrageen, tragacanth, gum arabicum, alginates, pectins, polyoses, guar meal, carob bean meal, starch, dextrines, gelatins, casein, cellulose ether, hydroxyethyl- and hydroxypropyl cellulose derivatives, polyacryl- and polymethacryl compounds, vinyl polymers, polycarboxylic acids, polyethers, polyimines, polyamides, polysilicic acids, clay minerals, zeolithe, and silicic acids.

Preferred hydrocolloids of the invention are, for example, methyl celluloses, which are called the methyl ethers of the cellulose. They are characterized by the following structural formula 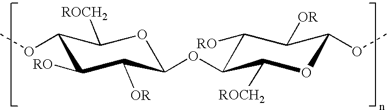
where R can be hydrogen or a methyl group.

Particularly advantageous for the purposes of the present invention are the mixed cellulose ethers, also generally named methyl celluloses, which contain besides a dominating content of methyl in addition 2-hydroxyethyl, 2-hydroxypropyl, or 2-hydroxybutyl groups. Particularly preferred are (hydroxypropyl)methyl celluloses, for example, those available from Dow Chemical Comp. under the trade name Methocel E4M.

Also advantageous in accordance with the invention is sodium carboxylmethyl cellulose, the sodium salt of the glycolic acid ether of the cellulose, for which R in the structural formula I can be hydrogen and/or CH2—COONa. Particularly preferred is sodium carboxymethyl cellulose, also named cellulose gum, which is available from Aqualon under the trade name Natrosol Plus 330 CS.

Also preferred for the purposes of the present invention is xanthan (CAS No. 11138-66-2), also named xanthan gum, which is an anionic heteropolysaccharide that is normally formed by fermentation from corn sugar and isolated as potassium salt. It is produced from Xanthomonas campestris and some other species under aerobic conditions with a molecular weight from 2×10⁶ to 24×10⁶. Xanthan is formed from a chain with beta-1,4-bonded glucose (cellulose) with side chains. The structure of the subgroups consists of glucose, mannose, glucoronic acid, acetate, and pyruvate. Xanthan is the name for the first microbial anionic heteropolysaccharide. It is produced from Xanthomonas campestris and some other species under aerobic conditions with a molecular weight of 2-15 106. Xanthan is formed from a chain with beta-1,4 bonded glucose (cellulose) with side chains. The structure of the subgroups consists of glucose, mannose, glucoronic acid, acetate, and pyruvate. The number of the pyruvate units determines the viscosity of the xanthan. Xanthan is produced in two-day batch cultures with a yield of 70% to 90% based on the applied carbon hydrate. In this process, yields of 25 to 30 g/l are obtained. Processing occurs after destroying the culture by precipitation with, for example, 2-propanol. Xanthan is subsequently dried and ground.

An advantageous gel former for the purposes of the present invention is also carrageen, a gel-forming extract structured in the same way as agar from North Atlantic red algae (chondrus crispus and gigartina stellata) of the florideae.

Often, the name carrageen is used for the dried algae product and carrageenan for the extract from same. The carrageen that is precipitated from the hot water extract is a colorless to sand-colored powder with a molecular weight range from 100,000 to 800,000 and a sulfate content of about 25%. Carrageen is easily soluble in warm water; when being cooled, a thixotropic gel forms even when the water content is 95-98%. The firmness of the gel is caused by the double-helix structure of the carrageen. In the case of carrageenan, one distinguishes between three main constituents; the gel-forming K-fraction consists of D-galactose-4-sulfate and 3,6-anhydro-α-D-galactose, which are alternately linked in a glycosidic way in the 1,3- and 1,4-positions (in comparison, agar contains 3,6-anhydro-α-galactose). The non-gelling gamma-fraction is composed of 1,3-glycosidically linked D-galactose-2-sulfate and 1,4-linked D-galactose-2,6-disulfate residues, and is easily soluble in cold water. The I-carrageenan structured in 1,4 linkage from D-galactose-4-sulfate in 1,3-linkage and 3,6-anhydro-α-D-galactose-2-sulfate in a 1,4 linkage is both water-soluble and gel forming. Further carrageen types are likewise identified by Greek characters: α, β, γ, μ, ν, ξ, π, ω, χ. Likewise, the type of present cations (K⁺, NH₄⁺, Na⁺, Mg²⁺, Ca²⁺) influences the solubility of the carrageens.

Polyacrylates are likewise gelators that are to be advantageously used for the purposes of the present invention. Advantageous polyacrylates in accordance with the invention are acrylate-alkylacrylate-copolymers, in particular those, which are selected from the group of the so-called carbomers or carbopols (Carbopol® is actually a registered trademark of NOVEON Inc.) In particular, the advantageous acrylate-alkylacrylate-copolymer or copolymers according to the invention is or are characterized by the following structure 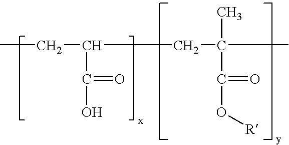
where R′ is a long-chain alkyl residue and x and Y are numbers that symbolize the respective stoichiometric component of the respective comonomers.

Preferred in accordance with the invention are acrylate-copolymers and/or acrylate-alkylacrylate-copolymers, which are available, for example, under the trade names Carbopol® 1382, Carbopol® 981, Carbopol® 980, and Carbopol 5984®, Aqua SF-1 from NOVEON Inc., or Aculyn® 33 from International Specialty Products Corp.

Also advantageous are copolymers from C10-30-alkylacrylates and one or more monomers of the acrylic acid, methacrylic acid, or their esters, which are crosslinked with allyl ether or the saccharose or allyl ether of pentaerythritol

Advantageous are compounds, which bear the INCl description “acrylates/C10-30 alkyl acrylates cross polymer.” Particularly advantageous are those that are available under the trade names Permulen TR1 and Permulen TR2 from NOVEON Inc.

Also advantageous are compounds with the INCl description “acrylates/C12-14 pareth-25 acrylate copolymer” (available under the trade name Synthalen® W2000 from 3V Inc.), with the INCl description “acrylates/steareth-20 methacrylate copolymer” (available under the trade name Aculyn® 22 from International Specialty Products Corp.), with the INCl description “acrylates/steareth-20 itaconate copolymer” (available under the trade name Structure 2001® from National Starch), and with the INCl description “acrylates/aminoacrylates/C10-30 alkyl PEG-20 itaconate copolymer” (available under trade name Structure Plus® from National Starch), and similar polymers.

The particularly preferred hydrocolloids of the invention are: acrylates copolymer (AQUA SF-1), acrylates/C10-30 alkyl acrylate crosspolymer (Carbopol ETD 2020), xanthan gum (Kelter).

The preparations of the invention advantageously contain one or more hydrocolloids/gel formers in a concentration from 0.1 to wt. %, preferably 0.2 to 6 wt. %, and more preferably 0.3 to 4 wt. % based on the total weight of the preparation.

Film Formers

According to the invention, the preparation may contain one or more film formers. According to the invention advantageous film formers can be selected from the compounds listed in the table.

| | | | Example |
|---|---|---|---|
| INCI Name | CAS-Number | Polymer Type | (Trade Name) |
| Polyquaternium-2 | CAS 63451-27-4 | Urea, N,N′-bis[3- | Mirapol ® A-15 |
| | | (dimethylamino)pro- | |
| | | pyl]-, polymer with 1, | |
| | | 1′-oxy bis(2- | |
| | | chloroethane) | |
| Polyquaternium-5 | CAS 26006-22-4 | Acrylamide, β- | |
| | | methacryloxyethyltri- | |
| | | ethylammoniummeth- | |
| | | osulfate | |
| Polyquaternium-6 | CAS 26062-79-3 | N,N-Dimethyl-N-2- | Merquat ® 100 |
| | | propenyl-2-propen- | |
| | | aminium chloride | |
| Polyquaternium-7 | CAS 26590-05-6 | N,N-Dimethyl-N-2- | Merquat ® S |
| | | propenyl-2-propen- | |
| | | aminium chloride, 2- | |
| | | propenamide | |
| Polyquaternium-10 | CAS 53568-66-4, | Quaternary | Celquat ® SC- |
| | 55353-19-0, 54351- | ammonium salt of | 230M |
| | 50-7, 68610-92-4, | hydroxyethyl | |
| | 81859-24-7 | cellulose | |
| Polyquaternium-11 | CAS 53633-54-8 | Vinylpyrrolidone/di- | Gafquat ® 755 |
| | | methylaminoethyl- | N |
| | | methacrylate- | |
| | | copolymer/diethylsul- | |
| | | fate reaction product | |
| Polyquaternium-16 | CAS 29297-55-0 | Vinylpyrrolidone/vi- | Luviquat ® |
| | | nylimi-dazolinium | HM552 |
| | | methochloride | |
| | | copolymer | |
| Polyquaternium-17 | CAS 90624-75-2 | | Mirapol ® AD-1 |
| Polyquaternium-19 | CAS 110736-85-1 | Quaternized water- | |
| | | soluble polyvinyl | |
| | | alcohol | |
| Polyquaternium-20 | CAS 110736-86-2 | Water-dispersible | |
| | | quaternized | |
| | | polyvinyloctadecyl | |
| | | ether | |
| Polyquaternium-21 | | Polysiloxane- | Abil ® B 9905 |
| | | polydimethyl- | |
| | | dimethylammonium | |
| | | acetate copolymer | |
| Polyquaternium-22 | CAS 53694-17-0 | Dimethyldiallyl | Merquat ® 280 |
| | | ammonium | |
| | | chloride/acrylic acid | |
| | | copolymer | |
| Polyquaternium-24 | CAS 107987-23-5 | Polymeric | Quartisoft ® |
| | | quaternary | LM-200 |
| | | ammonium salt of | |
| | | the hydroxyethyl | |
| | | cellulose | |
| Polyquaternium-28 | CAS 131954-48-8 | Vinylpyrrolidone/meth- | Gafquat ® HS- |
| | | acrylamidopropyl- | 100 |
| | | tri-methyl | |
| | | ammonium chloride | |
| | | copolymer | |
| Polyquaternium-29 | CAS 92091-36-6, | Chitosan that was | Lexquat ® CH |
| | 148880-30-2 | reacted with | |
| | | propylene oxid and | |
| | | quaternized with | |
| | | epichlorohydrin | |
| Polyquaternium-31 | CAS 136505-02-7, | Polymeric, | Hypan ® QT |
| | 139767-67-7 | quaternary | 100 |
| | | ammonium salt, | |
| | | which is produced | |
| | | by reacting DMAPA- | |
| | | acrylate/acrylic | |
| | | acid/acrylonitrogen | |
| | | copolymers and | |
| | | diethylsulfate | |
| Polyquaternium-32 | CAS 35429-19-7 | N,N,N-trimethyl-2- | |
| | | {[82-methyl-1-oxo-2- | |
| | | propenyl)oxy]- | |
| | | ethanaminium | |
| | | chloride, polymer | |
| | | with 2-propenamide | |
| Polyquaternium-37 | CAS 26161-33-1 | | |
| Polyquaternium-44 | | copolymers | |
| | | quaternary | |
| | | ammonium salt from | |
| | | vinylpyrrolidone and | |
| | | quaternized | |
| | | imidazoline | |

Further advantageous film formers are cellulose derivatives and quarternized guar gum derivatives, in particular guar hydroxypropylammonium chloride (for example, Jaguar Excel®, Jaguar C162® from Rhodia, CAS 65497-29-2, CAS 39421-75-5).

It is also possible and advantageous to use as film formers nonionic poly-N-vinylpyrrolidone/polyvinylacetate copolymers (for example, Luviskol VA 64W®, BASF), anionic acrylate copolymers (for example, Luviflex soft®, BASF), and/or amphoteric amide/acrylate/methacrylate copolymers (for example, Amphomer®, National Starch).

Complexing Agents

It is also advantageous to add complexing agents to the preparation of the invention. The complexing agents are advantageously selected from the group consisting of ethylenediaminetetraacetic acid (EDTA) and its anions, nitrilotriacetic acid (NTA) and its anions, hydroxyethylenediamineftriacetic acid (HOEDTA) and its anions, diethylenepentaacetic acid (DPTA) and its anions, tetrasodium iminodisuccinates, trisodium ethylenediamine disuccinates.

Lipids

An oil component of the cosmetic or dermatological preparation that may be desired for the purposes of the present invention, is advantageously selected from the group of esters of saturated and/or unsaturated, branched and/or unbranched alkane carboxylic acids having a chain length of 3 to 30 C atoms, and saturated and/or unsaturated, branched and/or unbranched alcohols having a chain length of 3 to 30 carbon atoms from the group of the esters of aromatic carboxylic acids, and saturated and/or unsaturated, branched and/or unbranched alcohols having a chain length of 3 to 30 C atoms. Such ester oils may then be advantageously selected from the group of isopropyl myristate, isopropyl palmitate, isopropyl stearate, isopropyl oleate, n-butylstearate, n-hexyl laurate, n-decyl oleate, isooctyl stearate, isononyl stearate, inononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, as well as synthetic, semi-synthetic, and natural mixtures of such esters, for example, jojoba oil.

The oil component may also be advantageously selected from the group of branched and unbranched hydrocarbons and hydrocarbon waxes, the silicone oils, the dialkyl ethers, the group of the saturated or unsaturated, branched or unbranched alcohols, as well as the fatty acid triglycerides, namely the triglycerol esters of saturated and/or unsaturated, branched and/or unbranched alkane carboxylic acids having a chain length from 8 to 24, in particular 12 to 18 carbon atoms. The fatty acid triglycerides may advantageously be selected, for example, from the group of the synthetic, semisynthetic, and natural oils, for example, olive oil, sunflower seed oil, soy oil, peanut oil, rape oil, almond oil, palm oil, coconut oil, palm kernel oil, and the like.

Any mixtures of such oil and wax components can also be used advantageously for the purposes of the present invention.

Advantageously, the oil component is selected from the group of 2-ethylhexyl stearate, octyldodecanol, isotridecyl isononanoate, isoeicosane, 2-ethylhexyl cocoate, C₁₂₋₁₅-alkyl benzoate, triglycerides of the caprylic-capric acid, dicaprylyl ether.

Especially advantageous are mixtures of C₁₂₋₁₅ alkyl benzoate and 2-ethylhexyl isostearate, mixtures of C₁₂₋₁₅ alkyl benzoate and isotridecyl isononanoate, as well as mixtures of C₁₂₋₁₅ alkyl benzoate, 2-ethylhexyl-isostearate and isotridecyl isononanoate.

Of the hydrocarbons, paraffin oil, squalane and squalene are to be used with advantage for the purposes of the present invention.

Advantageously, the oil component can also include a content of cyclic or linear silicone oils or completely consist of such oils, although it is preferred to use an additional content of other oil phase components apart from the silicone oil or silicone oils.

Advantageously, cyclomethicone (for example, octamethylcyclotetrasiloxane) is used as silicone oil in accordance with the invention. However, other silicone oils can also be used advantageously for the purposes of the present invention, for example, hexamethylcyclotrisiloxane, polydimethylsiloxane, poly(methylphenylsiloxane).

Particularly advantageous are also mixtures of cyclomethicone and isotridecyl isononanoate, as well as such of cyclomethicone and 2-ethylhexyl isostearate.

Furthermore, the oil component is advantageously selected from the group of the phospholipids. Phospholipids are acylated phosphoric ester glycerols. Of greatest importance among the phosphatidylcholines are, for example, the lecithins, which are characterized by the general structure 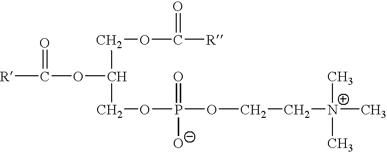
where R′ and R″ typically represent unbranched aliphatic residues with 15 or 17 carbon atoms and up to 4 cis-double bonds.
Aqueous Phase

Advantageously, an aqueous phase of the invention may also contain water-soluble ingredients, for example, low-carbon alcohols, diols or polyols, as well as ethers thereof, preferably ethanol, isopropanol, propylene glycol, glycerol, ethylene glycol, ethylene glycol monoethyl- or -monobutyl ether, propylene glycol monomethyl, -monoethyl-, or -monobutyl ether, diethylene glycol monomethyl or -monoethyl ether, and analogous products, furthermore alcohols of a low carbon number, for example, ethanol, isopropanol, 1,2-propanediol, methylpropanediol, and glycerol.

According to the invention, the cosmetic of the preparation of the invention, which is contained in the dispenser that is used in accordance with the invention, is present in the form of an emulsion, an aqueous solution, or as a waterfree formulation.

According to the invention, a preparation of the invention may advantageously contain besides one or more aqueous phases one or more oil phases, and be present in the form of W/O, O/W, W/O/W, or O/W/O emulsions. Preferably, such formulations may also be a microemulsion (for example, a PIT emulsion), solids emulsion (i.e. an emulsion that is stabilized by solids, for example a Pickering emulsion). Particularly preferred in accordance with the invention are transparent or translucent microemulsions.

According to the invention, the preparation can also be present in the form of an emulsion or dispersion. In this connection it is possible and advantageous for the purposes of the present invention to use both macroemulsions and microemulsions, O/W emulsions, W/O emulsions, S/W emulsions, W/S emulsions, as well as multiphase emulsions.

According to the invention, the compositions may contain besides the foregoing substances, additional substances as are customary in cosmetics, for example, perfume, dyes, antimicrobial agents, refatting agents, complexing and sequestering agents, pearlescent agents, plant extracts, selftanners (for example, DHA), depigmentors, antidandruff ingredients, vitamins, additional active ingredients, complexes of gamma-oryzanol and calcium salts, niacinamide and its derivatives, panthenol and its derivatives, subtilisin, mineral, preservatives, bactericides, pigments, which have a coloring effect, thickeners, softeners, moisturizers, and/or humectants, or other customary constituents of a cosmetic or dermatological formulation, such as alcohols, polyols, polymers, foam stabilizers, electrolytes, organic solvents or silicone derivatives. The pH value of the preparations according to the invention is adjusted in a customary manner for the person of skill in the art with the corresponding acids (lactic acid, citric acid, phosphoric acid, etc.), and bases (for example, NaOH).

In accordance with the invention the preparation of the cosmetic according to the invention or the dispenser used in accordance with the invention has a viscosity of at least 500 mPas.

Within the scope of the present disclosure, the referenced viscosity values of the preparations and individual substances were determined with the aid of a viscometer of the type VT 02 Viscotester by Haake (temperature: 25° C., rotational body diameter 24 mm, rotor speed 62.5 rpm).

II. The Dispenser of the Invention

To accomplish the objects that form the basis of the invention, the initially described dispenser (this is the dispenser disclosed in EP-A-0 230 252) is further developed to the extent that the delivery device comprises a delivery element that is adapted for axial displacement relative to the container and the head section. This delivery element comprises a delivery plunger adapted for sliding displacement in the delivery chamber, with the delivery plunger connecting to a delivery shaft that circumferentially surrounds a delivery channel, which comprises a delivery channel inlet opening communicating with the delivery chamber and a delivery channel outlet opening that can be brought by displacing the delivery element relative to the head section, to a position, in which the delivery channel outlet opening opens toward the dispensing channel.

In the case of the dispenser according to the invention, the delivery chamber opens toward the dispensing channel via a delivery channel outlet opening, which is exposed by axially displacing the delivery element relative to the head section. This relative movement is preferably realized in that the head section is manually actuated, i.e., slidably displaced in the axial direction toward the container. Accordingly, the passage of the pasty product from the delivery chamber to the product discharge opening at the end of the dispensing channel is already opened by a translational movement of the head section relative to the delivery element. A previous pressure buildup in the delivery chamber as has been necessary in the generic art for opening the passage, is no longer required. Accordingly, the actuation forces for dispensing pasty products from the dispenser are reduced.

In the case of the dispenser according to the invention, the delivery chamber is followed by a delivery channel, which is surrounded by a delivery shaft. At the end of this delivery channel, the pasty product that is removed from the delivery chamber, is discharged through the delivery channel outlet opening into the dispensing channel. Only after discharging the product from the delivery channel outlet opening is same available in the dispensing channel.

The remaining dispensing channel is at any rate shorter than in the customarily used dispensers. Accordingly, a clearly lesser volume of pasty substance is affected by possible oxidation processes. This makes it possible to shorten the remaining residual length of the dispensing channel in particular in the case of such products that are extremely susceptible to oxidation, in that the dispensing channel opens toward the outside in the extension of the end side of the head section.

In an advantageous configuration of the dispenser according to the invention, the delivery channel outlet opening is recessed on the circumferential surface of the delivery shaft and mounts on the head section a bushing that covers the delivery channel outlet opening in the starting position of the delivery device, so that a lifting movement of the head section for dispensing the pasty substance results in a simple manner in an exposure of the delivery outlet opening in that the delivery shaft is moved relative to the bushing. This preferred configuration is not only simple, but also permits arranging the delivery channel outlet opening in the direct vicinity of the inlet opening of the delivery channel for the product being delivered.

Taking into account a satisfactory axial guidance of the delivery device relative to the head section, the above-described bushing is preferably constructed as a guide bushing for the delivery device, and has at least one guide surface that cooperates with the circumferential surface of the delivery shaft.

With respect to a forced closing of the delivery channel outlet opening when returning the head section to its starting position, a preferred further development of the present invention proposes to provide on the head section and on the delivery device entraining means that entrain the delivery device upon manual actuation when returning the head section to its starting position.

Preferably, the foregoing entraining means are formed by an entraining shoulder formed on the bushing, which cooperates with an entrainment rim that is made integral with the delivery shaft. This entrainment rim is preferably formed on the delivery shaft on its end side, so that the delivery channel outlet opening that is recessed below the entrainment rim can be sealed in the starting position in that the entrainment rim lies against the walls of the head section.

In the foregoing preferred configuration, the volume accumulating in the dispensing channel can be further reduced in that the entrainment shoulder is formed at the end of the bushing and in the transition zone to the dispensing channel, and that the entrainment rim extends in the end side region of the delivery channel which is closed on its end side, as is proposed in a preferred further development of the invention. In this preferred configuration, a shaft cap arranged on the end side of the delivery shaft covers the dispensing channel in the starting position of the delivery device in substantially flush relationship, and preferably includes the entrainment rim.

According to a preferred embodiment of the present invention, the delivery plunger is actuated preferably via the end surfaces of the guide bushing. In this preferred further development, the delivery plunger extends beyond the delivery shaft in the radial direction for forming an annular abutment surface for a pressure surface, which is formed on the end side of the guide bushing, and which is arranged in the starting position in axially spaced relationship with the abutment surface, and is caused to lie against the abutment surface by axially displacing the head section in the direction toward the container.

Likewise with respect to a constructional simplification, a further preferred configuration of the present invention proposes to form the inner wall of the delivery chamber by an inner sleeve, which is provided on the end side of the container facing the head section. In this configuration, the inner sleeve projects beyond the end side of the container on the side facing the head section. Preferably, the inner sleeve is made integral with the container for purposes of reducing components.

For a simple centering of the head section while assembling the dispenser, and an easy mounting of the head section on the container, a mating head section is proposed according to a preferred development of the present invention. This mating head section comprises a retaining cylinder, which is slipped like a cup over the abovementioned inner sleeve, and a guide cylinder, which is arranged in concentric relationship with the retaining cylinder and controls the sliding displacement of the head section. The guide cylinder and/or the retaining cylinder allow/allows an easy concentric alignment of the head section relative to the cylinder. Furthermore, the guide cylinder improves the guidance of the lifting movement of the head section during an actuation of the dispenser.

In the case of a further preferred configuration of the dispenser of the invention, wherein the end of the guide cylinder on the side of the delivery chamber forms a stop for the delivery plunger, the delivery plunger is guided relatively lengthwise on the one hand, and the displacement of the delivery plunger is limited in a simple manner on the other hand. Such a displacement limitation secures, for example, the head section in the starting position on the container when the entrainment means are operatively connected.

On its side facing the bottom, the retaining cylinder preferably has an annular shoulder which forms an abutment surface for a helical spring that biases the head section in the starting position. This provides the advantage that the outer circumferential surface of the retaining cylinder encloses the helical spring on the inside and thus prevents the spring from buckling. In the case of this preferred configuration, the annular shoulder is positioned on the end side of the container and is thus especially suited for securing the mating head section in the axial direction relative to the container.

According to a further, particularly preferred configuration, the mating head section and the head section are formed as a prefabricated dispenser component. In this case, it is particularly preferred to slip the head section and the mating head section with their outer lateral surfaces over each other in the fashion of cups. The mating head section has at least one stop for limiting the axial displacement of the head section relative to the mating head section. In the case of such a configuration, a restoring element, for example the abovementioned helical spring which keeps the head section and the mating head section biased in axially spaced relationship, is preferably located in the interior space that is enclosed by the lateral surfaces. The abovementioned stop limits the axial displacement of the head section, i.e., following the assembly of the head section and mating head section with the inclusion of the spring, it ensures that the two components, which can be displaced relative to each other, are held together. The resulting dispenser component can be positioned on containers of different configurations, which allows a cost-effective production of the dispenser for a great variety of applications and container volumes.

A particularly simple and durable connection between the prefabricated dispenser component and the container is formed in that the dispenser component engages the container via latching means formed on the mating head section and the end side of the container.

In the case of the dispenser of the invention, the head section is preferably lengthwise displaceable such that it can be moved, in a first step, by means of manual actuation from the starting position by a first axial distance in order to butt against the delivery plunger, while simultaneously exposing the delivery channel outlet opening to the dispensing channel, to a center position, and that it can then be moved, while continuing its axial displacement and entraining the delivery plunger, from the center position into a final dispensing position, in which the delivery chamber, as a result of displacing the delivery plunger, has reached its smallest volume. In the case of this preferred configuration, the operations of exposing the delivery channel outlet opening and compressing the substance in the delivery channel occur within the scope of an equidirectional movement of the head section in the direction toward the container. This preferred configuration permits a constructionally simple solution for the dispenser of the invention, wherein the head section acts directly upon the delivery plunger and drives the latter, after opening the delivery channel outlet opening for delivering the pasty substance. This movement of the head section usually takes place against the force of a biasing element, for example, a spring, which ensures that upon relieving the head section, the latter pushes away from the container and the final dispensing position. During this movement, the axial distance a is covered in a first step, i.e. the delivery channel outlet opening is again closed. During this closing movement, the delivery shaft and the dispensing channel move relative to one another, which results in an increase in the volume of the dispensing channel at its inlet. The pasty substance located in the dispensing channel is thus pulled back in the direction toward the pump chamber, i.e. it is removed from the product discharge opening of the dispensing channel in the head section.

According to a preferred configuration of claims 15-18, a sealing component is located at this product discharge opening. The sealing component is preferably of such a nature that it opens in order to discharge the pasty product because of a pressure difference between the dispensing channel and the atmosphere. When—as aforsaid—the pasty substance in the dispensing channel is pulled back away from the product discharge opening, a relative negative pressure will result in the dispensing channel, which ensures that the sealing component seals the product discharge opening in a particularly effective manner.

With respect to a best possible sealing, it is preferred to form the product discharge opening around a closing pin arranged in the dispensing channel.

Preferably, this closing pin is made integral with the head section. The likewise annular sealing component has a sealing lip which can be positioned for sealing on the closing pin. While it closes in the case of an active negative pressure the dispensing channel in an effective manner, it opens for discharging the pasty product, a comparatively large product discharge opening, which permits delivering the product with a relatively low pressure loss.

A highly effective sealing component can be formed in a particularly cost-effective manner on the head section by means of a two-component injection molding process, as is proposed according to a preferred development of the present invention. In the case of this configuration, the sealing component is secured to the head section. The sealing component is preferably formed from a flexible plastic, more preferably from a thermoplastic elastomer. It has been found that it is possible to seal the product discharge opening in an effective manner, in particular with the use of a thermoplastic elastomer.

It has been found that the material for the sealing part can be utilized in a particularly preferred manner for forming a functional surface on the end-side outer surface of the head section. Such a functional surface may be, for example, a pushing surface which improves the haptic properties, and which the user of the dispenser pushes when using it. Such a functional surface is preferably formed by a coating at least on the end side of the exterior of the head section. The sealing component and the coating are made integral, preferably by of a two-component injection molding process after producing the head section by an injection molding process.

Further details, advantages and features of the present invention can be taken from the following description of an embodiment in conjunction with the drawing, in which:

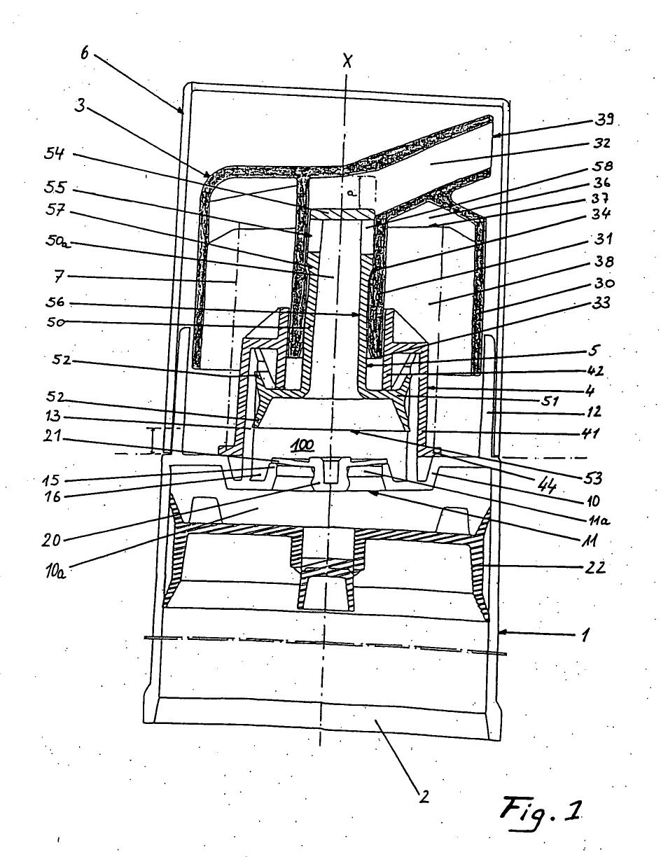

FIG. 1 is an axially sectioned view of a first embodiment of a dispenser according to the invention; and

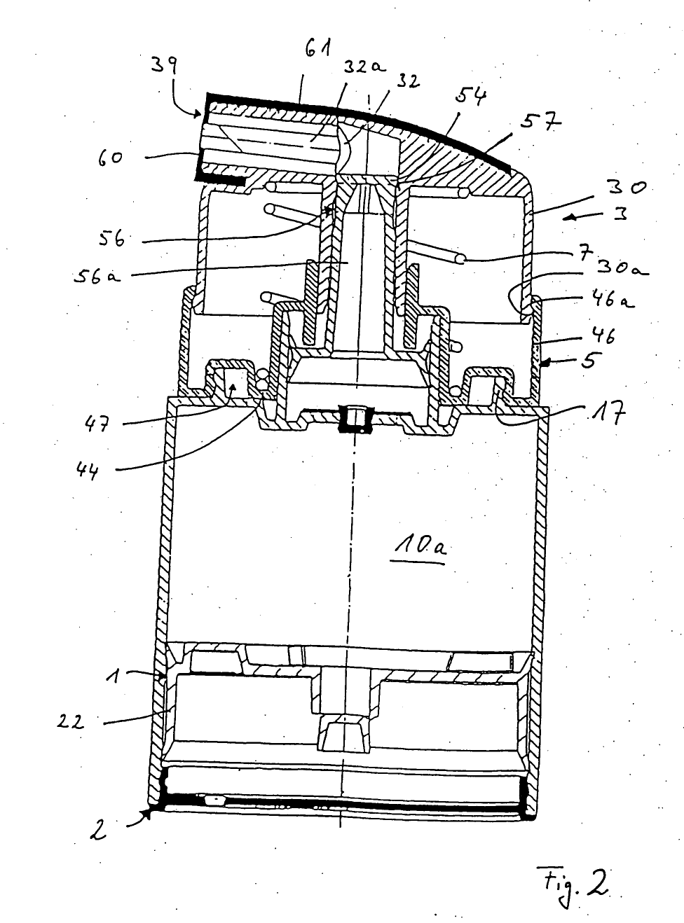

FIG. 2 is an axially sectioned view of a second embodiment of the dispenser according to the invention.

The embodiment of a dispenser of the invention as shown in FIG. 1 comprises a cup-shaped container 1 which connects on its underside to a bottom plate 2 that is latched to the container 1. On its other, upper end side, the container 1 comprises a cover 10, which includes a container opening 11. On its side facing away from the container 1, this cover 10 is formed for accommodating a dispenser head that comprises a head section 3, a mating head section 4 and a pressure plunger 5. The dispenser also comprises a sealing cap 6 that is slipped over an outer sleeve 12 of the container 1, with the outer sleeve 12 extending above the cover 10. The container 1, the bottom plate 2, the mating head section 4, and the pressure plunger 5 are designed as rotationally symmetrical components and are arranged in concentric relationship with a longitudinal center axis X. Located between the head section 3 and the mating head section 4 is a schematically indicated helical spring 7 which biases the head section 3 relative to the mating head section 4 in the starting position shown in FIG. 1.

The head section 3 has a cylindrical outer shell 30, which is arranged radially within, and directly adjacent to, the outer sleeve 12 of the container 1 and in concentric relationship with same. The outer sleeve 12 of the container 1 axially projects beyond the container end of the outer shell 30. Accordingly, the embodiment of the dispenser shown in FIG. 1 appears, even with the sealing cap removed, as a closed unit comprising the container 1 and the head section 3. As is explained in greater detail in the following, the head section 3 and the pressure plunger 5 are held for axial displacement relative to the container 1, with the pressure plunger 5 being moreover axially displaceable relative to the head section 3.

The cylindrical wall of the container 1 encloses an interior 10a for accommodating the cosmetic or dermatological preparation. Retaining crosspieces 11a extend in star shape in the container opening 11. On the side of the cover 10 facing away from the interior 10a, a cylindrical inner sleeve 13 is arranged in concentric relationship with the container opening 11, with an outer sleeve 12 projecting axially beyond the inner sleeve 13, and the latter enclosing a delivery chamber 100. The inner wall of the inner sleeve 13 is smooth. The bottom of the delivery chamber 100 is formed by the cover 10 of the container 1. The cover 10 has an annular rim 15 which projects into the delivery chamber 100, encloses the container opening 11, and forms an annular gap 16 between itself and the inner sleeve 13.

The pressure plunger 5 comprises an essentially cylindrical, internally hollow delivery shaft 50 with a delivery plunger 51 made integral with its one end. The delivery plunger 51 radially projects beyond the delivery shaft 50 and mounts on its outer circumferential surface top and bottom sealing lips 52 which project in the axial direction beyond the essentially annular delivery plunger 51. On its end side facing the delivery shaft 50, the delivery plunger 51 forms an annular abutment surface.

At its one end, the delivery shaft 50 has a delivery channel inlet opening 53 which is recessed in the center of the annular delivery plunger 51. At its other end, the delivery shaft 50 is closed on the end side by a shaft cap 54. The shaft cap 54 covers a cylinder section 55 of the delivery shaft 50, which has a larger diameter than the remaining shaft region 56. An obliquely outwardly inclined entrainment rim 57 is located between this shaft region 56 and the cylinder section 55. Between the entrainment rim 57 and the shaft cap 54, a plurality of delivery channel outlet openings 58 are distributed over the outer circumferential surface of the cylinder section 55. Retaining crosspieces, which mount the shaft cap 54, extend in the circumferential direction between the delivery channel outlet openings 58. The delivery channel inlet opening 53 communicates with the delivery channel outlet openings 58 via a delivery channel 50a that is enclosed by the delivery shaft 50, and forms a delivery passage for the pasty substance which is free of non-return valves.

The head section 3 has a cylindrical outer shell 30 with an internally hollow guide bushing 31 arranged in concentric relationship therewith. This guide bushing communicates with a dispensing channel 32. The end of the guide bushing 31 forms a pressure surface 33 on its lower end side, with the outer shell 30 axially projecting beyond it. Adjacent to the end-side pressure surface 33, the guide bushing 31 has a first bushing section, which has a smaller inside diameter than a second bushing section, which follows the first section in the delivery direction of the pasty substance. Formed between the first and the second bushing sections is an entrainment shoulder 34, which interconnects the two sections with different diameters via a slope. The second bushing section ends in a dispensing channel 32, which projects laterally from the longitudinal center axis X.

Approximately at right angles with the longitudinal center axis X, the head section 3 comprises spring-abutment surfaces 37 that are formed on ribs 36. The ribs 36 extend approximately in the shape of a star from the bushing 31 to the inner surface of the outer shell 30. Accordingly, an annular space 38 which opens toward the underside of the head section 3 is formed between the inner surface of the outer shell 30, the outer surface of the guide bushing 31 and the spring-abutment surfaces 37.

The head section 3 opens toward the container side of the outer shell 30. Above this end side, it is essentially constructed in the fashion of a cap. A product discharge opening 39 of the dispensing channel 32 is located on the upper side of the head section 3 facing away from the end side of the outer shell 30.

The mating head section 4 has essentially two concentric cylinder sections, namely an outer retaining cylinder 41 and a guide cylinder 42 with a smaller diameter. The retaining cylinder 41 projects beyond the guide cylinder 42 on the side facing the container 1, whereas the guide cylinder 42 projects beyond the retaining cylinder 41 on the other side. On the end side of the retaining cylinder 41 facing away from the container 1, an annular crosspiece is provided which extends from there radially inward and butts approximately in the center against the outer surface of the guide cylinder 42.

The retaining cylinder 41 has an outwardly projecting, peripheral annular shoulder on its end side facing the container. The end side of the guide cylinder 42 that faces the container forms a stop for delivery plunger.

In the assembled state, the delivery plunger 51 of the pressure plunger 5 is located for sliding displacement in the inner sleeve 13 of the container 1 and thus covers the delivery chamber 100 on the end side. The mating head section 4 is arranged in concentric relationship with the inner sleeve 13 and slipped with its retaining cylinder 41 in a cup-like manner over the inner sleeve 13. The annular shoulder of the mating head section 4 butts against the end side of the cover 10 facing away from the container 1.

The annular shoulder of the mating head section 4 is located approximately in the region of the end side of inner sleeve 13. The radially inwardly adjoining guide cylinder 42 encloses the end of the guide bushing 31 of the head section 3. Located radially inside this guide bushing 31 is the delivery shaft 50 with its shaft region 56 having a smaller diameter. The delivery plunger 51 of the pressure plunger 5 is arranged for sliding displacement along the inner wall of the inner sleeve 13. The annular abutment surface of the delivery plunger 51 lies against the end side of the delivery-plunger stop of the guide cylinder 42. This retains the biasing force exerted by the spring 7 on the head section 3, which biases, via the abutment of the entrainment shoulder 34 and entrainment rim 57, the pressure plunger 5 in a direction away from the container 1.

Located between the delivery chamber 100 and the interior 10a of the container 1 is a container valve 20 which is designed in a manner known per se, which lies by way of its sealing washer 21 against the annular rim 15 of the cover 10, and seals the interior 10a relative to the delivery chamber 100.

When not in use, the dispenser is located in a starting position (0). During the use of the dispenser, a user pushes the head section 3 in the direction of the container 1. Because of the incompressibility of the substance contained in the delivery chamber 100 and the delivery channel 50a, the pressure plunger 5 remains in its position. The head section 3 moves relative to the pressure plunger 5 in the direction of the container 1. The form-fitting abutment between the entrainment rim 57 and the entrainment shoulder 34 is released until the shaft cap 54 abuts the inner surface of the bushing head 35 or—depending on the configuration—the end-side pressure surface 33 at the end of the guide bushing 31 comes to lie against the annular abutment surface 51a of the delivery plunger 51 (center position M). Following this axial displacement by the distance a, the delivery channel outlet openings 58 are exposed to the dispensing channel 32.

As is common in any other axial relative movement between the head section 3 and the mating head section 4 and/or between the head section 3 and the container 1, the head section 3 is slidably guided by the abutment of the outer circumferential surface of the guide bushing 31 against the inner circumferential surface of the guide cylinder 42. The relative movement between the head section 3 and the pressure plunger 5 is guided via the abutment of the circumferential surface of the second shaft section against the shaft region 56.

As the pushing movement of the head section 3 continues in the direction of the container 1, the pressure plunger 5 is caused to move along. The volume of the delivery chamber 100 decreases here, with the result that the pasty product located downstream of the container valve 20, as seen in the conveying direction, is discharged via the delivery channel outlet opening 58 in the dispensing channel 32. The pasty product leaves the dispensing channel via its product discharge opening 39.

At the end of this relative movement of the head section 3 in the direction of the container 1, the sealing lips 52 of the pressure plunger 5 on the side toward the container abut the end side of the annular gap 16. In this final dispensing position V, the delivery chamber 100 has reached its smallest volume.

When the user then releases the head section 3, the helical spring 7 pushes the head section 3 back in the opposite direction. In this process, the pressure plunger 5 remains for the time being in its final dispensing position V. Only the head section 3 does move away from the container 1, until the entrainment shoulder 34 comes to lie against the entrainment rim 57.

During this axial displacement by the distance a, the pasty product located in the dispensing channel 32 is pulled back into the space formed between the shaft cap 54 and the inside of the bushing head 35. Thereafter, at the end of this displacement, the pasty product lies no longer directly against the product-dispensing opening 39 of the dispensing channel 32, whereby it is avoided that upon completion of the delivery operation, the pasty product drips from the dispensing channel 32, or is adversely affected by soiling in the region of the product-dispensing opening 39.

Following the displacement by the distance a and the abutment of the entrainment rim 57 and entrainment shoulder 34, the pressure plunger 5 is also moved back, as the movement of the head section 3 continues, in the direction toward the starting position, which is reached when the delivery-plunger stop butts against the annular abutment surface of the delivery plunger 51. During the relative movement of the pressure plunger 5 away from the container 1, the pasty product is delivered from the interior 10a of the container 1, through the container opening 11, into the delivery chamber 100. The relative negative pressure produced in the interior 10a here results, in a manner known per se, in a follow-up movement of the follow-up plunger 22 located in the interior 10a.

FIG. 2 shows a second exemplary embodiment of the dispenser of the invention.

In this embodiment, like parts which correspond to those of the foregoing embodiment have been provided with the same numerals. The container 1 of the exemplary embodiment which is shown in FIG. 2 is made essentially identical with the above-described container, with an outer container wall that encloses an interior 10a in which a follow-up plunger 22 is arranged for axial displacement, and which is closed by a bottom plate 2. In contrast to the above-described exemplary embodiment, the container 1 comprises a peripheral latching ring 17 on its end-side cover. The mating head section 4 is extended radially outward beyond the annular shoulder 44 and has a cylindrical outer wall 46 which extends essentially parallel to the retaining cylinder 51, and whose diameter is larger than the diameter of the outer shell 30 of the head section 3. Between the outer wall 46 and the retaining cylinder 41, the underside of the mating head section 4 facing the container includes a matching recess 47, which interacts with the latching ring 17 for a latching engagement between the mating head section 4 and the container 1.

In the case of the embodiment which is shown in FIG. 2, the mating head section 4 forms together with the head section 3 a prefabricated dispenser component. The free end of the outer wall 46 of the mating head section 4 facing away from the container 1, is angled radially inward to form a latching nose 46a, and axially projects beyond an annular bead 30a, which is provided on the outside of the outer shell 30 of the head section 3. This results in the formation of a stop which connects the mating head section 4 in captive fashion to the head section 3. This stop retains the spring forces applied by the spring 7. The dispenser component comprising the head section 3 and the mating head section 4 can thus be preassembled before being fitted onto the container 1. For this purpose, the spring 7 is inserted into the hollow space between the head section 3 and the mating head section 4. The two components 3, 4 are axially pushed into each other until the annular bead 30a has slid past the inwardly bent end of the outer wall 46.

In the case of the embodiment shown in FIG. 2, the pressure plunger 5 has an entrainment rim 57 which is made integral with the shaft cap 54. Accordingly, in the case of the starting position shown in FIG. 2, the entrainment rim 57 seals the dispensing channel 32. The delivery shaft 50 has a shaft region 56 of a reduced diameter, whose axial extension corresponds to the axial distance a. Accordingly, the axial displaceability of the pressure plunger 5 relative to the head section 3 is defined by the shaft cap 54, on the one hand, and the axial extension of the shaft region 56 with the reduced diameter, on the other hand.

The embodiment shown in FIG. 2 further differs from the previously described first embodiment in that the dispensing channel 32 contains a closing pin 32a which is made integral with the head section 3. The use of the closing pin 32a makes the product discharge opening 39 annular. In the illustrated embodiment, the product discharge opening 39 is covered by an annular sealing component 60 which connects to the head section 3 as a separate component made of a thermoplastic elastomer. In the starting position of FIG. 2, the sealing component 60 lies against the outer circumferential surface and against parts of the end side, in particular, however, the circumferential surface of the closing pin 32a, and thus seals the dispensing channel 32. A coating 61 is made integral with the sealing component 60. This coating is made of the same material as the sealing component 60 and extends over a large part of the cover on the end side of the head section 3. The coating 61 forms a non-slip functional surface on the head section 3.

When the dispenser shown in FIG. 2 is actuated, the same steps proceed as have been described in the foregoing, in particular with reference to FIG. 1. However, there is a difference from the aforesaid embodiment, primarily in that when the pressure plunger 5 and head section 3 are returned, the dispensing channel is sealed relative to the surroundings. During a movement of the pressure plunger 5 moving relative to the head section 3 in the direction toward the container 1, the product located in the dispensing channel 32—as has already been mentioned above—is pulled back into the interior of the head section 3 against the delivery direction. In the case of the embodiment illustrated in FIG. 2, the pressure gradient which is produced between the atmosphere and the dispensing channel 32 results in that the sealing component 60 lies in a totally sealable manner against the surfaces of the closing pin 32a. Accordingly, the pasty product staying in the dispensing channel 32 remains virtually unaffected by possible oxidation processes. In addition, the shaft cap 54 seals the delivery channel 50a relative to the dispensing channel 32, so that it is by all means avoided that in particular any pasty product being in the delivery channel 50a is adversely affected by air which may enter the dispensing channel 32.

The two embodiments described in the foregoing have both the advantage that the delivery channel openings 58 are exposed to the dispensing channel 32 only after a relative movement between the head section 3 and the pressure plunger 5. To deliver the pasty product from the delivery chamber in the direction of the product discharge opening 39, it is not necessary to use the initially built-up internal pressure in the delivery chamber 100 for opening a downstream non-return valve in the conveying direction. Accordingly, the pasty product can be delivered by applying lesser force. The two above-described embodiments also have the advantage that the pasty product is pulled back in the dispensing channel 32 against the conveying direction after actuating the head section, with the embodiment of FIG. 2 having the admissible advantage that by virtue of the sealing component 60 sealably lying against the closing pin 32a, the pasty product contained in the dispenser is reliably protected against being adversely affected, for example, by oxygen in the air.

Nomenclature:

1 Container; 2 Bottom plate; 3 Head section; 4 Mating head section; 5 Pressure plunger; 6 Sealing cap; 7 Helical spring; 10 Cover; 10a Interior; 11 Container opening; 11a Retaining crosspiece; 12 Outer sleeve; 13 Inner sleeve; 15 Annular rim; 16 Annular gap; 17 Latching ring; 20 Container valve; 21 Valve washer; 22 Follow-up plunger; 30 Outer shell; 30a Annular bead; 31 Guide bushing; 32 Dispensing channel; 32a Closing pin; 33 Pressure surface; 34 entrainment shoulder; 36 Rib; 37 Spring-abutment surface; 38 Annular space; 39 Product discharge opening; 41 Retaining cylinder; 42 Guide cylinder; 44 Annular shoulder; 46 Outer wall; 46a Latching nose; 47 Latching recess; 50 Delivery shaft; 50a Delivery channel; 51 Delivery plunger; 52 Sealing lips; 53 Delivery channel inlet opening; 54 Shaft cap; 55 Cylinder section; 56 Shaft region; 57 Entrainment rim; 58 Delivery channel outlet opening; 60 Sealing component; 61 Coating; 100 Delivery chamber

In accordance with the invention, the cosmetic or the dispenser of the invention is used for cleansing and taking care of the skin and/or skin appendages. Skin appendages relate in particular to hair and nails.

In accordance with the invention, the use of a dispenser for pasty products with a substantially cylindrical container (1) accommodating a cosmetic and/or dermatological preparation, which comprises in its bottom region a follow-up plunger (22) that is adapted for sliding displacement under the pressure of the ambient atmosphere along an inner wall of the container, and which mounts at is upper end a head section (3) that is adapted for sliding displacement relative to the container (1), with the head section comprising a channel (32) for dispensing the product that can be connected in a communicating manner to the container (1), and acting upon a manually actuatable delivery device with a volume-variable delivery chamber (100) for the product, is characterized in that the delivery device comprises a delivery element (5) that is axially displaceable relative to the container (1) and the head section (3), and includes a delivery plunger (51) adapted for sliding movement in the delivery chamber (100), with the delivery plunger (51) connecting to a delivery shaft (50) that circumferentially surrounds a delivery channel (50a), which comprises a delivery channel inlet opening (53) and a delivery channel outlet opening (58) that can be moved by displacing the delivery element (5) relative to the head section (3) to a position, in which the delivery channel opening (58) opens toward the dispensing channel (32), for storing and dispensing cosmetic and/or dermatological preparations as have been described within the scope of the present disclosure.

In particular, the use of the preparation of the cosmetic according to the invention or the dispenser of the invention as shower gel, bath in the tub, or hair shampoo is in accordance with the invention.

According to the invention, it is advantageous to use the preparation of the cosmetic or the dispenser of the invention as skin cream, lotion, or gel for the care of the skin. In this connection, it is preferred according to the invention, that the cosmetic and/or dermatological preparation is a preparation for protecting the skin against sun light (sunscreen), or for the prophylaxis, treatment, and/or care of dry skin, unclean to oily skin, acne, aged skin, skin ageing, baby skin and/or neurodermatitis.

The products of the invention are also excellently suited for cleaning and taking care of objects of daily life (for example, dishes, table and cabinet surfaces, cars, clothing, laundry).

The following examples are to illustrate the present invention without limiting it. All specified quantities, moieties, and percentages are based on the weight and the total quantity or the total weight of the preparations, unless otherwise specified.

Exemplary Formulations for the Cosmetic
and/or Dermatological Preparation
O/W-Emulsions

| | Example |
|---|---|
| | 1 |
| Glyceryl stearate citrate | 2.0 |
| Stearyl alcohol | 2.0 |
| Cetyl alcohol | 1.0 |
| Myristyl myristate | 1.0 |
| Hydrated coconut fat glycerides | 2.0 |
| Butyleneglycol dicaprylate/dicaprate | 1.0 |
| Ethylhexyl coconut fatty acid ester | 3.0 |
| Vaseline | 1.0 |
| Dicaprylyl ether | 3.0 |
| TiO₂ | 1.0 |
| Ethylhexylmethoxycinnamate | 2.0 |
| Ubiquinone (Q10) | 0.03 |
| Panthenol | 1.0 |
| Biotin | 0.05 |
| Phenoxy ethanol | 0.8 |
| Alkyl ester of p-hydroxybenzoic acid | 0.6 |
| Cyclodextrin | 0.4 |
| Xanthan gum | 0.2 |
| Polyacrylic acid (carbomer) | 0.05 |
| Glycerol | 15.0 |
| Water- and/or oil-soluble dyes | 0.05 |
| Fillers/additives (distarch phosphate, | 0.1 |
| SiO₂, BHT, talcum) | |
| Perfume | q.s. |
| Water | ad 100 |
| [symbol] | |
| | Example |
| | 2 |
| Glycerylstearate, | 5.0 |
| selfemulsifying | |
| Stearyl alcohol | 1.0 |
| Shea butter | 1.0 |
| C12-15 Alkyl benzoate | 3.0 |
| Caprylic acid/capric acid triglyceride | 1.0 |
| Mineral oil | 1.0 |
| Dicarprylylcarbonate | 3.0 |
| Ethylhexylmethoxycinnamate | 3.0 |
| Ethylhexyltriazone | 1.0 |
| Bis-ethylhexyloxyphenol- | 1.0 |
| methoxyphenyltriazine | |
| Niacinamide | 0.2 |
| Citric acid, sodium salt | 0.1 |
| Creatine | 0.8 |
| Phenoxyethanol | 0.6 |
| Alkyl ester of p-hydroxybenzoic acid | 0.3 |
| (Paraben) | |
| Hexamidindiisethionate | 0.04 |
| 1,3-dimethylol-5,5-dimethyl- | 0.1 |
| hydantoin(DMDM Hydantoin) | |
| Ethanol (denatured) | 2.0 |
| Ammoniumacryloyldimethyltaurate/ | 0.5 |
| vinylpyrrolidone copolymers | |
| Glycerol | 10.0 |
| Butylene glycol | 10.0 |
| Fillers/additives (distarch phosphate, | 1.0 |
| SiO₂, talcum) | |
| Perfume | q.s. |
| Water | ad 100 |
| | Example |
| | 3 |
| Glyceryl stearate | 3.0 |
| PEG-40-stearate | 1.0 |
| Cetyl alcohol | 3.0 |
| Shea butter | 2.0 |
| C12-15 alkyl benzoate | 2.0 |
| Caprylic acid/capric acid triglyceride | 2.0 |
| Octyldodecanol | 1.0 |
| Vaseline | 1.0 |
| Cyclomethicone | 4.0 |
| Dimethicone | 1.0 |
| Dicaprylyl ether | 2.0 |
| TiO₂ | 1.0 |
| Ethylhexylmethoxycinnamate | 5.0 |
| Butylmethoxydibenzoylmethane | 2.0 |
| 2-hydroxy-4-methoxy-benzophenone | 2.0 |
| (Oxybenzone) | |
| Bisabolol | 0.3 |
| Tocopheryl acetate | 1.0 |
| Allantoin | 0.5 |
| Phenoxyethanol | 0.3 |
| Alkyl ester of p-hydroxybenzoic acid | 0.2 |
| (Paraben) | |
| Diazolidinyl urea | 0.1 |
| Cyclodextrin | 0.3 |
| Xanthan gum | 0.1 |
| Ammoniumacryloyl dimethyltaurate/ | 0.3 |
| vinylpyrrolidone copolymers | |
| Glycerol | 7.0 |
| Fillers/additives (distarch phoshate, | 0.2 |
| SiO₂, talcum) | |
| Perfume | q.s. |
| Water | ad 100 |
| | Example |
| | 4 |
| Glyceryl stearate | 1.0 |
| Stearic acid | 4.0 |
| Behenyl alcohol | 1.0 |
| Cetyl alcohol | 3.0 |
| Butyleneglycol Dicaprylate/Dicaprate | 1.0 |
| Caprylic acid/capric acid triglyceride | 2.0 |
| Jojoba oil | 1.0 |
| Dimethicone | 1.0 |
| Dicarprylylcarbonate | 3.0 |
| Ethylhexylcyanodiphenylacrylate | 5.0 |
| (Octocrylene) | |
| bis-Ethylhexyloxyphenol- | 2.0 |
| methoxyphenyltriazine | |
| 2-hydroxy-4-methoxy-benzophenone | 3.0 |
| (Oxybenzone) | |
| Phenylbenzimidazole sulfonic acid | 2.0 |
| Cyclodextrin | 0.5 |
| Retinol | 0.05 |
| Phenoxyethanol | 0.2 |
| Alkyl ester of p-hydroxybenzoic acid | 0.3 |
| (Paraben) | |
| Iodopropinylbutylcarbamate | 0.02 |
| Ammoniumpolyacryloyldimethyltaurate | 0.3 |
| Urea | 2.0 |
| Glycerol | 5.0 |
| Butylene glycol | 1.0 |
| Fillers/additives (distarch phosphate, | 0.5 |
| SiO₂, talcum) | |
| Perfume | q.s. |
| Water | ad 100 |
| | Example |
| | 5 |
| Glyceryl stearate | 2.0 |
| PEG-40-stearate | 1.0 |
| Myristyl myristate | 1.0 |
| Cetearyl alcohol | 2.0 |
| Shea butter | 2.0 |
| C12-15 alkylbenzoate | 3.0 |
| Caprylic acid/capric acid triglyceride | 2.0 |
| Ethylhexyl ester of coconut fatty acid | 1.0 |
| Vaseline | 2.0 |
| Cyclomethicone | 5.0 |
| TiO₂ | 1.0 |
| Ethylhexylmethoxycinnamate | 3.0 |
| 2-Hydroxy-4-methoxy-benzophenone | 2.0 |
| (Oxybenzone) | |
| □-Glycosylrutin | 0.2 |
| Tocopherol | 0.5 |
| Genistein | 0.02 |
| Retinylpalmitate | 0.1 |
| Sodium ascorbylphosphate | 0.1 |
| Phenoxyethanol | 0.2 |
| Alkyl ester of p-hydroxybenzoic acid | 0.3 |
| (Paraben) | |
| Polyacrylic acid (carbomer) | 0.1 |
| Aluminum starch octenylsuccinate | 0.5 |
| Glycerol | 5.0 |
| Fillers/additives (distarch phosphate, | 0.05 |
| SiO₂, talcum) | |
| Perfume | q.s. |
| Water | ad 100 |
| | Example |
| | 6 |
| Cetyl alcohol | 2.0 |
| Shea butter | 1.0 |
| Caprylic acid/capric acid triglyceride | 2.0 |
| Octyldodecanol | 1.0 |
| Dicaprylylcarbonate | 5.0 |
| Dimethylpolysiloxane (Dimethicone) | 1.0 |
| Polydecene | 2.0 |
| Ethylhexylmethoxycinnamate | 3.0 |
| Bis-ethylhexyloxyphenol- | 0.5 |
| methoxyphenyltriazine | |
| Sodium ascorbylphophate | 0.05 |
| Iminodisuccinate | 0.2 |
| Dihydroxyacetone | 3.0 |
| Phenoxyethanol | 0.3 |
| Alkyl ester of p-hydroxybenzoic acid | 0.4 |
| (Paraben) | |
| Crosslinked alkylacrylate | 0.2 |
| (alkylacrylate crosspolymer) | |
| Glycerol | 5.0 |
| Perfume | q.s. |
| Water | ad 100 |

| | Example |
|---|---|
| | 7 |
| Glyceryl stearate | 2.5 |
| PEG-40-stearate | 1.0 |
| Cetearyl alcohol | 2.0 |
| Hydrogenated cocoglycerides | 1.0 |
| Shea butter | 2.0 |
| C12-15 alkylbenzoate | 4.0 |
| Caprylic acid/capric acid triglycerides | 2.0 |
| Octyldodecanol | 1.0 |
| Vaseline | 1.0 |
| Dicarprylylcarbonate | 3.0 |
| Octadecene dicarboxylic acid | 2.0 |
| TiO₂ | 1.0 |
| Ethylhexylcyanodiphenyl-acrylate | 5.0 |
| (Octocrylene) | |
| Phenylbenzimidazole-sulfonic acid | 1.0 |
| 2-Hydroxy-4-methoxy- benzophenone | 2.0 |
| (Oxybenzone) | |
| Ubiquinol | 0.03 |
| Iminodisuccinate | 0.2 |
| Phenoxyethanol | 0.5 |
| Alkyl ester of p-hydroxybenzoic acid | 0.4 |
| (Paraben) | |
| Iodopropinylbutylcarbamate | 0.05 |
| 2-Ethylhexylglycerol ether (octoxyglycerol) | 0.5 |
| Polyacrylic acid (carbomer) | 0.2 |
| Nylon microparticles | 1.0 |
| Glycerol | 10.0 |
| Additives (distarch phosphate, SiO₂, | 0.03 |
| talcum) | |
| Perfume | q.s. |
| Water | ad 100 |

| | Example |
|---|---|
| | 8 |
| Polyglyceryl-3-methylglucose distearate | 3.0 |
| Sorbitan stearate | 1.0 |
| Behenyl alcohol | 2.0 |
| Cetyl alcohol | 1.0 |
| C12-15 alkylbenzoate | 2.0 |
| Butylene glycol dicaprylate/dicaprate | 2.0 |
| Caprylic acid/capric acid triglycerides | 2.0 |
| Hydrogenated polydecene | 1.0 |
| Dimethylpolysiloxane (Dimethicone) | 1.0 |
| Dicarprylylcarbonate | 2.0 |
| Ethylhexylmethoxycinnamate | 5.0 |
| Butylmethoxydibenzoylmethane | 2.0 |
| Daidzein | 0.05 |
| Genistein | 0.04 |
| Tocopheryl acetate | 0.5 |
| Iminodisuccinate | 0.2 |
| Phenoxyethanol | 0.4 |
| Alkyl ester of p-hydroxybenzoic acid | 0.4 |
| (Paraben) | |
| Denatured ethanol | 5.0 |
| Xanthan gum | 0.2 |
| Ammoniumacryloyldimethyltaurate/ | 0.3 |
| vinylpyrrolidone copolymer | |
| Glycerol | 4.5 |
| Additives (distarch phosphate, SiO₂, | 0.1 |
| talcum) | |
| Perfume | q.s. |
| Water | ad 100 |

| | Example |
|---|---|
| | 9 |
| Cetearylglucoside | 2.0 |
| Myristyl myristate | 1.0 |
| Stearyl alcohol | 4.0 |
| C12-15 alkylbenzoate | 2.0 |
| Caprylic acid/capric acid triglycerides | 3.0 |
| Hydrogenated polydecene | 1.0 |
| Dicarprylylcarbonate | 3.0 |
| Polydecene | 4.0 |
| Ethylhexylmethoxycinnamate | 3.0 |
| Ethylhexylcyanodiphenyl acrylate | 3.0 |
| (octocrylene) | |
| Butylmethoxydibenzoylmethane | 1.0 |
| Tocopherol | 1.0 |
| Trisodium EDTA | 0.1 |
| Phenoxyethanol | 0.7 |
| Alkyl ester of p-hydroxybenzoic acid | 0.4 |
| (Paraben) | |
| 2-Ethylhexylglycerolether (Octoxyglycerol) | 0.4 |
| Ammoniumpolyacryloyldimethyltaurate | 0.3 |
| Aluminium starch octenylsuccinate | 1.0 |
| Glycerol | 4.0 |
| Butylene glycol | 2.0 |
| Additives (distarch phosphate, SiO₂, | 3.0 |
| talcum) | |
| Perfume | q.s. |
| Water | ad 100 |

| | Example |
|---|---|
| | 10 |
| Polyethyleneglycol(21)stearyl-ether | 2.0 |
| (steareth-21) | |
| Polyethyleneglycol(2)stearyl-ether | 1.0 |
| (steareth-2) | |
| Cetearyl alcohol | 2.0 |
| Shea butter | 1.0 |
| C12-15 Alkylbenzoate | 5.0 |
| Octyldodecanol | 1.0 |
| Mineral oil | 1.0 |
| Octamethyltetrasiloxane (cyclomethicone) | 2.0 |
| Dicaprylylether | 2.0 |
| TiO2 | 1.0 |
| Ethylhexylmethoxycinnamate | 4.0 |
| Ethylhexyltriazone | 1.0 |
| Urea | 2.0 |
| Biotin | 0.02 |
| Trisodium EDTA | 0.2 |
| Phenoxyethanol | 0.5 |
| Alky ester of p-hydroxybenzoic acid | 0.3 |
| (Paraben) | |
| Iodopropinylbutylcarbamate | 0.1 |
| Xanthan gum | 0.3 |
| Polyacrylic acid (carbomer) | 0.1 |
| Ammoniumacryloyldimethyltau- | 0.3 |
| rate/vinylpyrrolidone copolymers | |
| Glycerol | 6.0 |
| Additives (distarch phosphate, SiO2, | 0.05 |
| talcum, aluminum stearate) | |
| Perfume | q.s. |
| Water | ad 100 |

W/O-Emulsions

| | Example |
|---|---|
| | 11 |
| Polyglyceryl-3 diisostearate | 1.5 |
| PEG-40 Sorbitan perisostearate | 2.5 |
| Lanolin alcohol | 0.3 |
| Mineral oil | 8.0 |
| Cera Microcrystallina | 2.5 |
| Octamethyltetrasiloxane (Cyclomethicone) | 4.0 |
| Isohexadecane | 2.0 |
| Isopropylpalmitate | 5.0 |
| Urea | 2.0 |
| Tocopheryl acetate | 1.0 |
| Iodopropinylbutylcarbamate | 0.1 |
| Magnesium sulfate | 0.6 |
| Lactic acid | 0.7 |
| Lactic acid, sodium salt | 1.5 |
| Glycerol | 7.0% |
| Perfume | q.s. |
| Desalted water | ad 100% |

| | Example |
|---|---|
| | 12 |
| PEG-30 dipolyhydroxy stearate | 3.0 |
| Caprylic/capric triglyceride | 1.5 |
| Mineral oil | 12.5 |
| Vitamin K | 0.1 |
| Na-Ascorbylphosphate | 0.1 |
| Phenoxyethanol | 0.5 |
| 2-Phenylbenzimidazol-5-sulfonic acid | 3.0 |
| Lactic acid | 0.7 |
| Lactic acid, sodium salt | 1.5 |
| Glycerol | 8.0 |
| Perfume | q.s. |
| Desalted water | ad 100 |

| | Example |
|---|---|
| | 13 |
| Cetyl dimethicone copolyol | 2.0 |
| Octamethyltetrasiloxane (cyclomethicone) | 5.0 |
| Dimethylpolysiloxane (dimethicone) | 5.0 |
| Phenyltrimethicone | 3.0 |
| Isohexadecane | 3.0 |
| Dimethiconol | 1.0 |
| Phenoxyethanol | 0.5 |
| Alkyl ester of p-hydroxybenzoic acid (Paraben) | 0.2 |
| Iodopropinylbutylcarbamate | 0.05 |
| Sodium chloride | 2.0 |
| Urea | 1.0 |
| Allantoin | 0.5 |
| Na-ascorbylphosphate | 0.1 |
| Butylene glycol | 3.0 |
| Glycerol | 6.0 |
| Perfume | q.s. |
| Desalted water | ad 100 |

1. A cosmetic or dermatological product comprising:
a) a dispenser for pasty products comprising:
a substantially cylindrical container comprising an inner wall and a follow-up plunger, said follow-up plunger located within said container and adapted for sliding displacement along the inner wall of said container under the pressure of the ambient atmosphere, and
a manually actuatable delivery device, said delivery device comprising:
a head section mounted on the upper end of said container, said head section adapted for sliding displacement relative to said container, said head section comprising a dispensing channel and
a delivery element adapted for axial displacement relative to said container and said head section, the delivery element comprising:
a volume-variable delivery chamber,
a delivery plunger that is adapted for sliding displacement in the delivery chamber, a delivery shaft, and
a delivery channel,
said delivery shaft circumferentially surrounding said delivery channel, and said delivery plunger connecting to said delivery shaft, wherein said delivery channel comprises a delivery channel inlet opening communicating with said delivery chamber and a delivery channel outlet opening, the delivery channel outlet opening being movable by displacing the delivery element relative to the head section from a starting position to an open position, in which the delivery channel outlet opening opens toward the dispensing channel; and
b) a cosmetic or dermatological preparation within said container and delivered through said delivery chamber and delivery shaft to said dispensing channel, said preparation comprising at least one ingredients having at least one property selected from the group consisting of I) sensitivity to oxidation with oxygen or air; II) sensitivity to hydrolysis with water or air moisture; III) irritability to the olfactory receptors of humans or animals; IV) the ability to causes a temperature change of the preparation under the influence of water, air moisture or oxygen; and V) the ability to loses its physiological effectiveness under the influence of water, air moisture, or oxygen.

2. The product as claimed in claim 1, wherein said preparation comprises said at least one ingredient in a total concentration from 0.01 to 5 wt. %, based on the total weight of the preparation.

3. The product as claimed in claim 1, wherein said at least one ingredient is selected from the group consisting of antioxidants, vitamins, enzymes, coenzymes, perfumes, fragrances, repellents, self-tanners, unsaturated lipids, oils, fats, waxes, polyphenols, enzymes, flavonoids, isoflavonoids, lignans, polyols, polyethylene glycols, and plant extracts which contain one or more of the foregoing active ingredients.

4. The product as claimed in claim 1, wherein said at least one ingredient is selected from the group consisting of vitamin A and its derivatives, vitamin B and its derivatives, vitamin C and its derivatives, vitamin E and its derivatives; vitamin F; polyphenols, ubiquinone Q10; ubiquinone Q10 in reduced form; 2,6-ditert.-butyl-4-methylphenol; dihydroxy acetone; niacinamide; pantothenic acid and its salts; panthenol; gamma-oryzanol; biotin; creatine, creatinine; subtilisin; alpha-glycosylrutin; allantoin; tannin; azulen; bisabolol; glycyrrhizin; hamamelin; urea; genistin; genistein; daidzin; daidzein; carnitine and its derivatives, and octadecene dicarboxylic acid.

5. The product as claimed in claim 1, wherein said preparation is present in the form of an emulsion, an aqueous solution, or a water-free formulation.

6. The product as claimed in claim 1, wherein said preparation has a viscosity of at least 500 mPa·s.

7. The product as claimed in claim 1, said cosmetic preparation further comprising a continuous aqueous phase.

8. The product as claimed in claim 7, said continuous phase comprising at least one hydrocolloid.

9. The product as claimed in claim 1, wherein said delivery channel outlet opening of the dispenser is recessed into the circumferential surface of said delivery shaft, and wherein said head section further comprises a bushing that covers the delivery channel outlet opening in the starting position of the delivery device.

10. The product as claimed in claim 9, wherein said bushing is constructed as a guide bushing that guides the delivery device for axial displacement, and wherein said bushing has at least one guide surface that interacts with the circumferential surface of the delivery shaft.

11. The product as claimed in claim 1, wherein said head section further comprises an entrainment means to entrain the delivery device upon manual actuation, when the head section is returned to the starting position.

12. The product as claimed in claim 11, wherein said entrainment means comprises an entrainment shoulder integral with the bushing, and an entrainment rim integral with the delivery shaft.

13. The product as claimed in claim 12, wherein said entrainment shoulder is provided on the end side of the bushing proximate said channel outlet opening, and that the entrainment rim is provided in the end side region of the delivery shaft.

14. The product as claimed in claim 10, wherein said delivery plunger radially projects beyond the delivery shaft forming an annular abutment surface, and wherein said wherein said guide bushing has on its end side a pressure surface, which is arranged in the starting position in axially spaced relationship with the abutment surface, and which is caused to lie against the abutment surface by axially displacing the head section in the direction toward the container.

15. The product as claimed in claim 1, wherein an inner wall of the delivery chamber is formed by an inner sleeve that is provided on the end side of the container facing the head section.

16. The product as claimed in claim 15, said dispenser further comprising a mating head section, comprising
a retaining cylinder in the shape of a cup over said inner sleeve, and
a guide cylinder that is arranged in concentric relationship with said retaining cylinder and controls the sliding displacement of said head section.

17. The product as claimed in claim 16, further comprising a stop for the delivery plunger, said stop located at the end of the guide cylinder facing the delivery chamber.

18. The product as claimed in claim 16, further comprising an annular shoulder on the side of the retaining cylinder facing a bottom of the container, said annular shoulder forming an abutment surface for a helical spring, said helical spring biasing the head section in the starting position, and placed on the end side of the container.

19. The product as claimed in claim 16, wherein said mating head section includes at least one stop for limiting the axial displacement of the head section, wherein said mating head section is integral with said head section as a prefabricated dispenser component and mounted to the end side of the container.

20. The product as claimed in claim 19, said dispenser further comprising a latching means formed on the mating head section and the end side of the container, said latching means used to engage said container with said mating head section.

21. The product as claimed in claim 1, wherein the said head section is axially displaceable such that the head section is movable in a first step, by means of manual actuation from the starting position, by a first axial distance, to an open position, wherein said head contacts the delivery plunger while simultaneously exposes the delivery channel outlet opening to the dispensing channel, and wherein subsequent to said first step the head section is movable, in a second step wherein said head section is further axially displaced from an open position to a final dispensing position, wherein said delivery plunger is entrained and said delivery chamber has reached its smallest volume as a result of displacing the delivery plunger.

22. The product as claimed in claim 1, said dispenser further comprising a sealing component arranged on the head section, which is used to seal a product discharge opening of the dispensing channel.

23. The product as claimed in claim 22, wherein the product dispensing opening is annular around a closing pin arranged in the dispensing channel, and said sealing component comprises a sealing lip that is made annular for lying in a sealable manner against the closing pin.

24. The product as claimed in claim 22, wherein said sealing component is formed from a flexible plastic material.

25. The product as claimed in claim 22, wherein said sealing component is made integral with a coating that is provided at least on the end side on the outer side of the head section.

26. A method of cleansing or taking care of the skin or skin appendages comprising:
a) providing a cosmetic or dermatological product comprising:
i) a dispenser for pasty products comprising:
a substantially cylindrical container an inner wall and a follow-up plunger, said follow-up plunger located within said container and adapted for sliding displacement along the inner wall of said container under the pressure of the ambient atmosphere, and
a manually actuatable delivery device, said delivery device comprising:
a head section mounted on the upper end of said container, said head section adapted for sliding displacement relative to said container, said head section comprising a dispensing channel that can be connected in a communicating manner to said container, and
a delivery element adapted for axial displacement relative to said container and said head section, the delivery element comprising:
a volume-variable delivery chamber
a delivery plunger that is adapted for sliding displacement in the delivery chamber,
a delivery shaft, and
a delivery channel,
said delivery shaft circumferentially surrounding said delivery channel, and said delivery plunger connecting to said delivery shaft,
wherein said delivery channel comprises a delivery channel inlet opening communicating with said delivery chamber and a delivery channel outlet opening, the delivery channel outlet opening being movable by displacing the delivery element relative to the head section from a starting position to a center position, in which the delivery channel outlet opening opens toward the dispensing channel; and
ii) a cosmetic or dermatological preparation within said container and delivered through said delivery chamber and delivery shaft to said dispensing channel, said preparation comprising at least one ingredient-having at least one property selected from the group consisting of I) sensitivity to oxidation with oxygen or air; II) sensitivity to hydrolysis with water or air moisture; III) irritates the olfactory receptors of humans or animals; IV) causes a temperature change of the preparation under the influence of water, air moisture or oxygen; and V) loses its physiological effectiveness under the influence of water, air moisture, or oxygen
b) axially sliding said head section toward said container to dispense said formulation to said dispensing channel, and
c) applying said formulation to the skin.

27. (canceled)

28. (canceled)

29. The product as claimed in claim 1, wherein said preparation comprises said at least one ingredient in a total concentration from 0.05 to 3 wt. %, based on the total weight of the preparation.

30. The product as claimed in claim 1, wherein said preparation comprises said at least one ingredient in a total concentration from 0.1 to 1 wt. %, based on the total weight of the preparation.

31. The product as claimed in claim 8, wherein said at least one hydrocolloid includes at least one hydrocolloid selected from the group consisting of polyacrylic acids and its derivatives, methacrylic acid and its derivatives, polysaccharide and its derivatives, and gums.

32. The product as claimed in claim 8, wherein said at least one hydrocolloid includes at least one hydrocolloid selected from the group consisting of cellulose and cellulose derivatives.

33. The product as claimed in claim 22, wherein said sealing component is formed from a thermoplastic elastomer.

34. The product as claimed in claim 1, wherein said at least one ingredient includes an ingredient sensitive to oxidation with oxygen or air.

35. The product as claimed in claim 1, wherein said at least one ingredient includes an ingredient sensitive to hydrolysis with water or air moisture.

36. The product as claimed in claim 1, wherein said at least one ingredient includes an ingredient having the property of irritability to the olfactory receptors of humans or animals.

37. The product as claimed in claim 1, wherein said at least one ingredient includes an ingredient having the ability to cause a temperature change of the preparation under the influence of water, air moisture, or oxygen.

38. The product as claimed in claim 1, wherein said at least one ingredient includes an ingredient having the ability to lose its physiological effectiveness under the influence of water, air moisture, or oxygen.
